(19) United States
(12) Patent Application Publication
(10) Pub. No.: US 2009/0123373 A1
(43) Pub. Date: May 14, 2009

(54) AMYLOID-IMAGING AGENTS

(76) Inventors: YANMING WANG, Beachwood, OH (US); Chunying Wu, Mayfield Heights, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/265,371

(22) Filed: Nov. 5, 2008

Related U.S. Application Data
(60) Provisional application No. 60/985,431, filed on Nov. 5, 2007.

(51) Int. Cl. A61K 49/00; C07D 413/10; A61K 51/04; A61K 49/10; A61K 49/04

(52) U.S. Cl. 424/1.89; 548/156; 424/9.1; 424/1.65; 424/1.85; 424/9.3; 424/9.44

(57) ABSTRACT

A molecular probe for use in the detection of amyloid in a subject includes a dibenzothiazole derivative.

### RELATED APPLICATION

This application claims priority from U.S. Provisional Application No. 60/985,431, filed Nov. 5, 2007, the subject matter, which is incorporated herein by reference.

### TECHNICAL FIELD

The present invention relates to molecular probes and to methods of their use, and particularly relates to molecular probes that can readily cross the blood brain barrier when systemically administered to a subject and selectively localize to amyloid in the subject's brain.

### BACKGROUND

Alzheimer's disease (AD) is a progressive and irreversible neurodegenerative disorder resulting in senile dementia that is characteristic of amyloid deposition in two types of brain lesions, senile plaques (SPs) and neurofibrillary tangles (NFTs) (Hardy and Higgins (1992) Science 256:184-185, Hardy and Selkoe (2002) Science 297, 353-356, Braak and Braak (1998) Neural. Transm. Suppl. 53: 127-140). Both SP and NFT accumulation have been suggested as early and specific events in the pathogenesis of AD (Selkoe (2000) Ann. N.Y. Acad. Sci.

924:17-25, Naslund et al (2000) JAMA 283:1571-1577, Selkoe (2000) JAMA 283: 1615-1617, Manczak et al. (2006) Hum. Mol. Genet. 15:1437-1449). Senile plaques are areas of disorganized neuropil with extracellular amyloid deposits at the center. Neurofibrillary tangles are intracellular deposits of tau protein consisting of two filaments twisted about each other in pairs.

Currently postmortem histopathological examination of SP and NFTs in the brain is still the only method for definitive diagnosis of Alzheimer's. One of the major tasks in AD research is to detect and quantify SP and NFT in living subjects, preferably at early or even pre-symptomatic stages. To date, applications of Positron Emission Tomography(PET) and Single Photon Emission Computer Tomography (SPECT) for amyloid imaging have been hampered by the lack of suitable amyloid-imaging probes.

Several types of amyloid-imaging agents have thus been synthesized and evaluated. Systematic modification of the dye Congeo Red resulted in a series of bisstyrylbenzene derivatives (Klunk et al. (1995) Neurobiol. Aging 16: 541-548, Styren et al. (2000) Histochem. Cytochem. 48:1223-1232, Link et al. (2001) Neurobiol. Aging 22:217-226, Ishii et al. (2002) Neurosci. Lett. 333:5-8, Mathis et al. (2004) Curr. Pharm. Des. 10:1469-1492, Han et al. (1996) J. Am. Chem. Soc. 118:4506-4507, Zhen et al. (1999) J. Med. Chem. 42: 2805-2815, Dezutter et al. (1999) Eur. J. Nucl. Med. 26:1392-1399, Zhuang et al. (2001) J. Med. Chem. 44: 1905-1914, Lee et al. (2002) J. Cereb Blood Flow Metab. 22:223-231). These bisstyrylbenzene derivatives exhibited high binding affinity and specificity with improved brain uptake. However, no lead compounds have been identified with in vivo pharmacokinetic profiles that meet a series of strict requirements set for in vivo imaging.

Further modification of CR also led to the design and synthesis of a series of stilbene derivatives as amyloid-imaging agents for either PET or SPECT studies (Zhuang et al. (2005) Nucl. Med. Biol. 32:171-184, Verhoeff et al. (2004) Am. J. Geriatri. Psychiatry 12:584-595, Ono et al. (2003) Nucl. Med. Biol. 30:565-571, Ono et al. (2005) Nucl. Med. Biol. 32:329-335, Kung et al. (2001) J. Am. Chem. Soc. 123:12740-12741, Zhang et al. (2005) Nucl. Med. Biol. 32:799-809, Zhang et al. (2005) J. Med. Chem. 48:5980-5988. Following appropriate radiolabeling, the stilbene derivatives have been evaluated for in vivo and in vitro binding properties to amyloid deposits and pharmacokinetic profiles. Most of these stilbene derivatives readily penetrated the blood-brain barrier and selectively bound to amyloid deposits at high affinities. These studies have led to the identification of a compound, termed [¹¹C]SB-13 ([¹¹C]-4-N-methylamino-4′-hydroxystilbene) that can be used for PET amyloid-imaging in human subjects (Verhoeff et al (2004)). In AD subjects, [¹¹C]SB-13 displayed an accumulate pattern that is considered consistent with the previously reported AD pathology. In contrast, little or no retention of [¹¹C]SB-13 was observed in age-matched control subjects (Verhoeff et al (2004)).

Another amyloid dye that has been extensively studied is thioflavin T (ThT). ThT is a positively charged histological dye for amyloid that cannot penetrate the BBB (Burns et al. (1967) Pathol. Bacteriol. 94:337-344). Elimination of the positive charge has led to the development of a series of benzothiazole and related heterocycles such as 2-aryl-substituted benzothiazole derivatives(Zhuang et al. (2001), Mathis et al. (2003) J. Med. Chem. 46:2740-2754, Mathis et al. (2002) Bioorg. Med. Chem. Lett. 12: 295-298, Wang et al. (2004) J. Mol. Neurosci. 24:55-62, Wang et al. (2003) J. Mol. Neurosci. 20:255-260, Wang et al. (2002) J. Mol. Neurosci. 19:11-16), 2-aryl-substituted benzooxazole derivatives (Zhuang et al. (2001) Nucl. Med. Biol. 28:887-894), 2-aryl-substituted benzofuran (Ono et al. (2002) Nucl. Med. Biol. 29:633-642), and imidazo[1,2-α]pyridine derivatives (Zhuang et al (2001) Nucl. Med. Biol. 28:887-894, Cai et al. (2004) J. Med. Chem. 47:2208-2218). Most of these neutral, lipophilic ThT analogs bind to amyloid fibrils with high affinity and specificity. The in vivo pharmacokinetic profiles of the above heterocyclic compounds have been extensively evaluated as potential amyloid-imaging agents. Compared to neutral CR analogs, lipophilic ThT analogs have even smaller molecular weights and display a higher brain uptake. A lead compound, termed PIB ([¹¹C]-2-(4-(methylamino)phenyl)-6-hydroxybenzothiazole), was thus identified for human studies. Extensive clinical PET studies indicated that PIB readily entered the brain and selectively bound to amyloid deposits in AD subjects. PIB accumulation is predominant in the cortical areas known for amyloid deposition in AD subjects. Conversely, PIB showed rapid entry and clearance in all cortical gray matter of healthy control subjects.

As an imaging agent for SPECT, a [123]-labeled imidazo[1,2-α]pyridine derivative, termed IMPY (6-iodo-2-(4′-dimethylamino-)phenyl-imidazo[1,2]pyridine), has been identified and its pharmacological effects have been evaluated in human subjects. Preliminary studies in both AD and normal control subjects demonstrated that IMPY is a safe radiotracer for clinical imaging studies (Newburg et al. (2006) J. Nucl. Med. 47:748-754). These studies paved the way for the potential use of [¹²³I] IMPY in clinical SPECT imaging of amyloid deposits in human subjects.

In addition, amyloid-imaging agents have also been derived from other histological dyes such as acridine orange (Suemoto et al. (2004) Neurosci. Res. 48:65-74, Shimadzu et al. (2003) 46:765-772), fluorine (Lee et al. (2003) Nucl. Med. Biol. 30:573-580), and DDNP (Agdeppa et al (2001) J. Neuroci. 21:RC189, Agdeppa (2003) Neurosci 117:723-730, Jacobson et al. (1996) J. Am. Chem. Soc. 118:5572-5579). In fact, the first PET amyloid-imaging studies in human subjects were carried out with an F-18-labeled DDNP analog termed [¹⁸ F] FDDNP ([¹⁸F]-2-(1-(2-(N-(2-fluoroethyl)-N-methylamino) naphthalene-6-yl)ethylidene)malononitrile) (FIG. 2) (Shoghi-Jadid (2002) Am. J. Geriatr. Psychiatry 10:24-35). Clinical studies suggested that [¹⁸F]FDDNP's retention in amyloid deposit regions may be due to selective binding to both SPs and NFTs in the brain.

### SUMMARY

The present invention relates to a molecular probe for use in the detection of amyloid of brain tissue of a subject. The molecular probe includes the general formula:

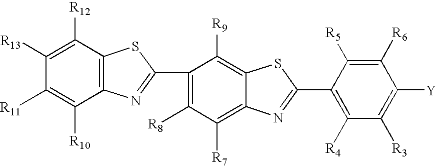

wherein Y is NR₁R₂, OR₂, or SR₂; each R₁-R₁₃ independently is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′ is H or a lower alkyl group), a tri-alkyl tin, a radiolabel, a chelating group, and a near infrared group; or pharmaceutically acceptable salts thereof. In an aspect of the invention, the benzothiazoles groups of the molecular probe are not quaternary amines.

In another aspect of the invention, at least one of R₁-R₁₃ includes a radiolabel. The radiolabel can be selected from the group consisting of ³H, ¹³¹I, ¹²³I, ¹²⁵I, ¹⁸F, ¹⁹F, ¹¹C, ⁷⁵Br, and ⁷⁶Br. In one example of a molecular probe in accordance with the present invention, R₃-R₁₂ can be H, and R₁₃ can be H or an electron donating group. In another example, R₃-R₁₂ can be H, and R₁₃ is selected from the group consisting of H, Cl, F, I, Br, a lower alkyl group, and OCH₃. In a further example, Y is NR₁R₂ and is selected from the group consisting of NH₂, NHCH₃, and N(CH₃)₂.

In a further aspect, the molecular probe can include a radiolabel, a chelating group or a near infrared imaging group and the amyloid can include at least one of amyloid deposits in senile plaques (SPs) and neurofibrillary tangles (NFTs) in a subject's brain tissue.

Another aspect of the invention relates to a molecular probe for use in the detection of amyloid of a subject that comprises the general formula:

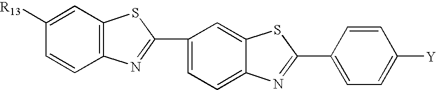

wherein Y is NR₁R₂, OR₂, or SR₂; each R₁, R₂, and R₁₃ independently is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′ is H or a lower alkyl group), a tri-alkyl tin, a radiolabel, a chelating group, and a near infrared group; or pharmaceutically acceptable salts thereof.

In an aspect of the invention, the radiolabel can be selected from the group consisting of ³H, ¹³¹I, ¹²³I, ¹²⁵I, ¹⁸F, ¹⁹F, ¹¹C, ⁷⁵Br, and ⁷⁶Br.

In one example of a molecular probe in accordance with the present invention, R₁₃ can be H or an electron donating group. In another example, R₁₃ is selected from the group consisting of H, Cl, F, I, Br, a lower alkyl group, and OCH₃. In a further example, Y is NR₁R₂ and is selected from the group consisting of NH₂, NHCH₃, and N(CH₃)₂. In a still further example, the molecular probe can have the following formula:

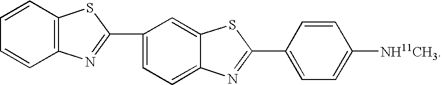

The present invention also relates to a method of detecting amyloid in an animal's brain tissue. The method includes administering to the tissue a molecular probe. The molecular probe can include the following general formula:

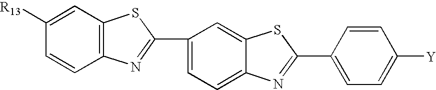

wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof. The binding of the molecular probe to the animal's brain tissue can then be detected.

In an aspect of the invention, the radiolabel can be selected from the group consisting of ³H, ¹³¹I, ¹²³I, ¹²⁵I, ¹⁸F, ¹⁹F, ¹¹C, ⁷⁵Br, and ⁷⁶Br.

In one example of a molecular probe in accordance with the present invention, R₁₃ can be H or an electron donating group. In another example, R₁₃ is selected from the group consisting of H, Cl, F, I, Br, a lower alkyl group, and OCH₃. In a further example, Y is NR₁R₂ and is selected from the group consisting of NH₂, NHCH₃, and N(CH₃)₂.

In another aspect of the invention, the molecular probe can be administered in vivo to the animal and be detected by an in vivo imaging modality. The imaging modality can include at least one of gamma imaging, Positron Emission Tomography (PET) imaging, micro Positron Emission Tomography (microPET) imaging, Single Photon Emission Computer Tomography (SPECT) imaging, magnetic resonance imaging, magnetic resonance spectroscopy, and near infrared imaging. The animal can be a human or a mouse and the molecular probe can be administered to the animal intravenously.

The present invention further relates to a method of detecting a neurodegenerative disorder in an animal. The method includes the first step of administering to the animal's brain tissue a molecular probe having the general formula:

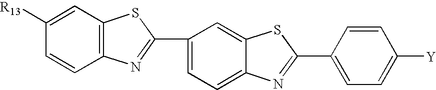

wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof. Following administration of the molecular probe, the animal's brain tissue is visualized using an imaging modality. The distribution of the molecular probe is correlated with neurodegenerative disorder in the animal.

The present invention still further relates to a method of monitoring the efficacy of a neurodegenerative disorder therapy in an animal. The method includes administering to the animal a molecular probe having the general formula:

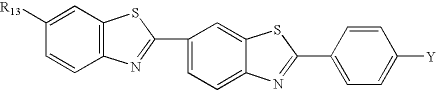

wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof. Following labeling amyloid in the animal's brain, the distribution of the molecular probe is visualized. The distribution of the molecular probe is then correlated with the efficacy of the neurodegenerative disorder therapy.

The present invention still further relates to a method of quantifying the amyloid load in an animal. The method includes administering to the animal a molecular probe having the general formula:

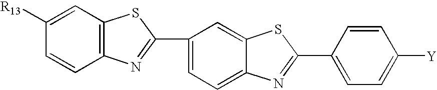

wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof. Following labeling amyloid in the animal's brain tissue, the distribution of the molecular probe is visualized. The distribution of the molecular probe is then correlated with the amyloid load in the animal.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1 illustrates plots showing the results of competitive binding assays of dibenzothiazole derivatives using [³H] PIB as the radioligand in AD brain tissue homogenates, and the binding affinity (Kᵢ) and lipophilicity (logP oct) of newly synthesized dibenzothiazole derivatives.

FIGS. 2A-2I illustrate images of in vitro staining of APPPSI mice brain sections and non-transgenic control brain sections with thioflavin-S, Pittsburgh imaging compound B (PIB), and molecular probes in accordance with the present invention.

FIG. 3 illustrates an image of amyloid deposition in Alzheimer disease (AD) human brain tissue sections stained using molecular probes in accordance with the present invention.

FIG. 4 illustrates a plot showing whole brain uptake of a ¹¹C labeled molecular probe in accordance with an aspect of the invention.

### DETAILED DESCRIPTION

The present invention relates to molecular probes that upon systemic administration (e.g., intravenous administration) to an animal in vivo can cross the blood-brain barrier and selectively localize to stain and/or bind to amyloid deposits or plaques (e.g., β-amyloid protein (Aβ)). The molecular probes can then be detected in vivo, ex vivo, or in vitro using conventional visualization techniques to indicate the presence or absence of amyloid deposits or plaques in the animal's brain tissue. The molecular probes of the present invention are non-quarternary amine derivatives of Thioflavin S, which can stain amyloid in tissue sections and bind to Aβ in vitro. The molecular probes of the present invention can be used to detect and stain amyloid in vivo in humans as well as other animals, such as mice. This is in contrast to molecular probes based on Thioflavin T derivatives, which do not localize to, stain, or bind to amyloid in mice. Staining of amyloid in vivo of both human and mice is advantageous because it allows the molecular probes of the present invention to be used in clinical assays for measuring and screening the efficacy of neurodegenerative disorder therapies (e.g., Alzheimer's therapies) and agents.

The molecular probes described herein may be used in methods of detecting amyloid in vivo, ex vivo, and in vitro in an animal's brain tissue, methods of detecting neurodegenerative disorders in an animal, methods of monitoring the efficacy of a neurodegenerative disorder therapy in an animal, and methods of quantifying the amyloid load in an animal.

In one aspect of the invention, the molecular probes can include lipophilic dibenzothiazole derivatives that have the following general formula:

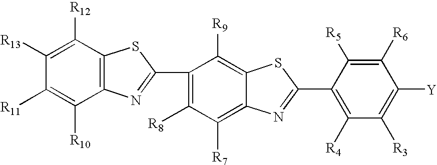

wherein Y is NR₁R₂, OR₂, or SR₂; each R₁-R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof. The benzothiazole groups in this aspect of the invention are not quarternary amines.

In another aspect of the present invention, the molecular probe can have the following general formula:

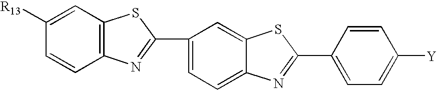

wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof.

In still another aspect of the present invention, the molecular probe can have the following formula:

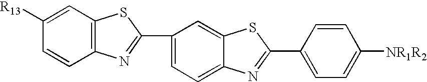

wherein each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a chelating group, and a near infrared group; or pharmaceutically acceptable salts thereof.

In yet another aspect of the present invention, the molecular probe can have the following formula:

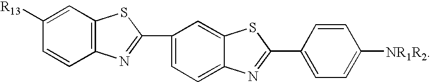

wherein each R₁, R₂, and R₁₃ independently is selected from the group consisting of H, F, Cl, Br, I, a lower alkyl group, (CH₂)ₙOR′(wherein n=1, 2, or 3); or pharmaceutically acceptable salts thereof.

By way of example, the molecular probe can be selected the group consisting of:

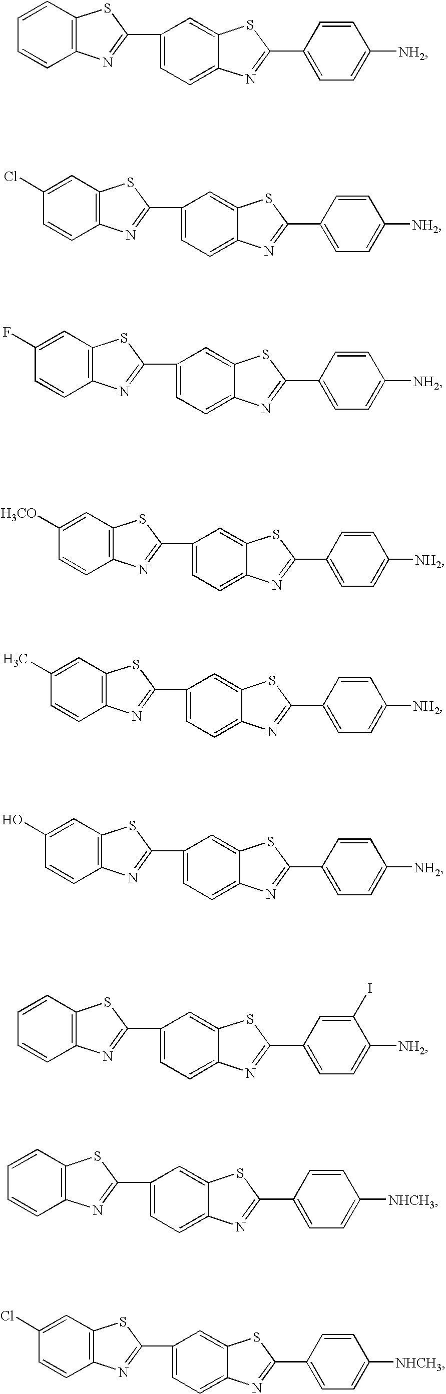

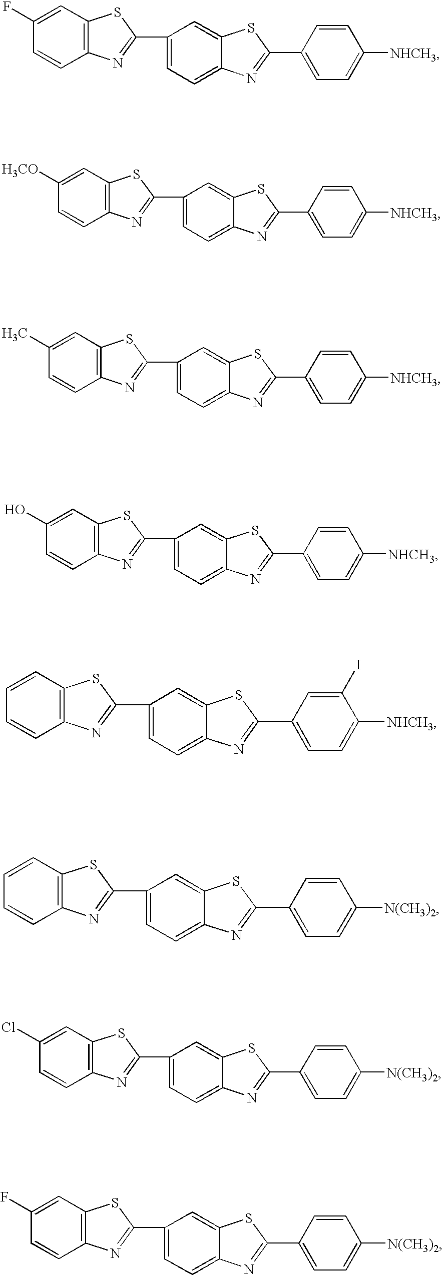

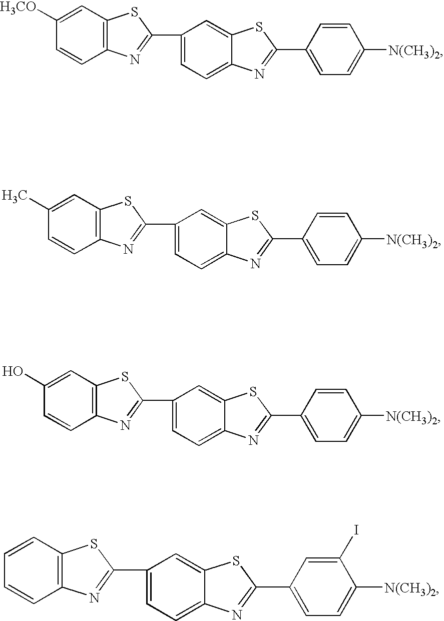

and pharmaceutically acceptable salts thereof.

The method of this invention determines the presence and location of amyloid deposits in an organ or body area, such as the brain, of a patient. The present method comprises administration of a detectable quantity of a pharmaceutical composition containing a molecular probe or a pharmaceutically acceptable water-soluble salt thereof, to a patient. A “detectable quantity” means that the amount of the detectable compound that is administered is sufficient to enable detection of binding of the compound to amyloid. An “imaging effective quantity” means that the amount of the detectable compound that is administered is sufficient to enable imaging of binding of the compound to amyloid.

The invention employs amyloid molecular probes which, in conjunction with non-invasive neuroimaging techniques, such as magnetic resonance spectroscopy (MRS) or imaging (MRI), or gamma imaging, such as positron emission tomography (PET) or single-photon emission computed tomography (SPECT), are used to quantify amyloid deposition in vivo. The term “in vivo imaging” refers to any method which permits the detection of a labeled molecular probe, as described above. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound.

For purposes of in vivo imaging, the type of detection instrument available is a major factor in selecting a given label. For instance, radioactive isotopes and ¹⁹F are particularly suitable for in vivo imaging in the methods of the present invention. The type of instrument used will guide the selection of the stable isotope. The half-life should be long enough so that it is still detectable at the time of maximum uptake by the target, but short enough so that the host does not sustain deleterious radiation. The radiolabeled compounds of the invention can be detected using gamma imaging wherein emitted gamma irradiation of the appropriate wavelength is detected. Methods of gamma imaging include, but are not limited to, SPECT and PET. For SPECT detection, the chosen radiolabel can lack a particulate emission, but will produce a large number of photons in, for example, a 140-200 keV range. For PET detection, the radiolabel can be a positron-emitting moiety, such as ¹⁹F.

In an aspect of the invention, the molecular probes can be used in conjunction with non-invasive neuroimaging techniques such as magnetic resonance spectroscopy (MRS) or imaging (MRI), positron emission tomography (PET), single-photon emission computed tomography (SPECT), and near infrared imaging. By way of example, the molecular probes can be labeled with ¹⁹F or ¹³C for MRS/MRI by general organic chemistry techniques known to the art. The molecular probes can also be radiolabeled with ¹⁸F, ¹¹C, ⁷⁵Br, or ⁷⁶Br for PET by techniques well known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The molecular probes can also be radiolabeled with ¹²³I, for SPECT by any of several techniques known to the art. See, e.g., Kulkarni, Int. J. Rad. Appl. & Inst. (Part B) 18: 647 (1991), the contents of which are hereby incorporated by reference. In addition, the molecular probes can be labeled with any radioactive iodine isotope, such as, but not limited to ¹³¹I, ¹²⁵I, or ¹²³I, by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art. See, Satyamurthy and Barrio J. Org. Chem. 48: 4394 (1983), Goodman et al., J. Org. Chem. 49: 2322 (1984), and Mathis et al., J. Labell. Comp. and Radiopharm. 1994: 905; Chumpradit et al., J. Med. Chem. 34: 877 (1991); Zhuang et al., J. Med. Chem. 37: 1406 (1994); Chumpradit et al., J. Med. Chem. 37: 4245 (1994).

The molecular probes can also be radiolabeled with known metal radiolabels, such as Technetium-99m (^{99m}Tc). Modification of the substituents to introduce ligands that bind such metal ions can be effected without undue experimentation by one of ordinary skill in the radiolabeling art. The metal radiolabeled molecular probes can then be used to detect amyloid deposits. Preparing radiolabeled derivatives of Tc^{99m} is well known in the art. See, for example, Zhuang et al., “Neutral and stereospecific Tc-99m complexes: [99 mTc]N-benzyl-3,4-di-(N2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

The methods of the present invention may use isotopes detectable by nuclear magnetic resonance spectroscopy for purposes of in vivo imaging and spectroscopy. Elements particularly useful in magnetic resonance spectroscopy include ¹⁹F and ¹³C.

Radioisotopes for purposes of this invention include beta-emitters, gamma-emitters, positron-emitters, and x-ray emitters. These radioisotopes include ¹³¹I, ¹²³I, ¹⁸F, ¹¹C, ⁷⁵Br, and ⁷⁶Br. Examples of stable isotopes for use in Magnetic Resonance Imaging (MRI) or Spectroscopy (MRS), according to this invention, include ¹⁹F and ¹³C. Examples of radioisotopes for in vitro quantification of amyloid in homogenates of biopsy or post-mortem tissue include ¹²⁵I, ¹⁴C, and ³H. Examples of radiolabels are ¹¹C or ¹⁸F for use in PET in vivo imaging, ¹²³I for use in SPECT imaging, ¹⁹F for MRS/MRI, and ³H or ¹⁴C for in vitro studies. However, any conventional method for visualizing diagnostic probes can be utilized in accordance with this invention.

In a specific example, a radiolabeled molecular probe of the present invention can have the following formula:

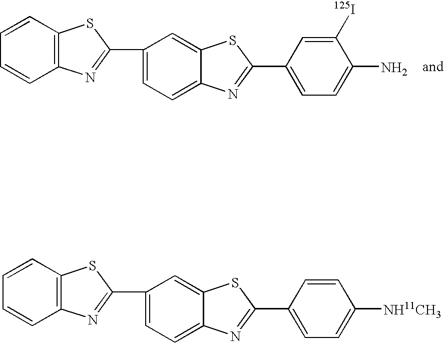

In another aspect of the invention, the molecular probe can be coupled to a chelating group (with or without a chelated metal group) to improve the MRI contrast properties of the molecular probe. In one example, as disclosed in U.S. Pat. No. 7,351,401 which is herein incorporated by reference in its entirety, the chelating group can be of the form W-L or V-W-L, wherein V is selected from the group consisting of —COO—, —CO—, —CH₂O— and —CH₂NH—; W is —(CH₂)ₙ where n=0, 1, 2, 3, 4, or 5; and L is:

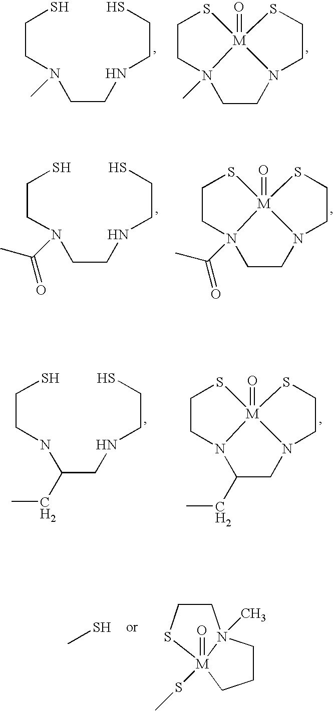

wherein M is selected from the group consisting of Tc and Re; or

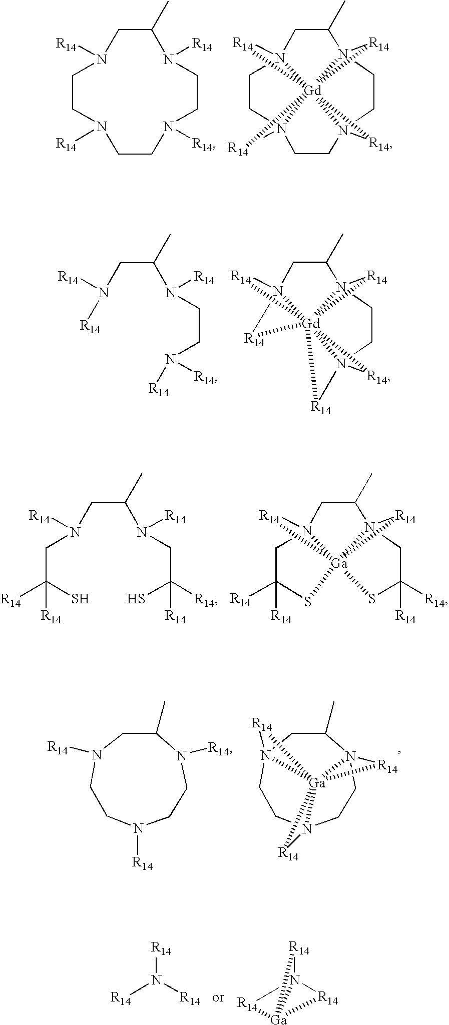

wherein each R₁₄ is independently is selected from one of:

### H,

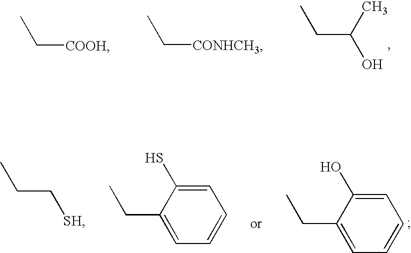

or an amyloid binding, chelating compound (with or without a chelated metal group) or a water soluble, non-toxic salt thereof.

The chelating group can be coupled to at least one benzothiazole group, benzene group, R₁-R₁₃ group, or be an R₁-R₁₃ group. In one example, the chelating group can be coupled to a terminal amino group through carbon a chain link. The carbon chain link can comprise, for example about 2 to about 10 methylene groups and have a formula of, for example, (CH₂)ₙ, wherein n=2 to 10.

In one example, the molecular probe with the chelating group can have the following formula:

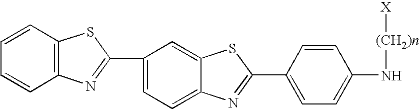

wherein X is a chelating group and n is 2 to 10; or a salt thereof.

In another embodiment, the molecular probe can be coupled to a near infrared group to improve the near infrared imaging of the molecular probe. Examples of near infrared imaging groups that can be coupled to the molecular probe include:

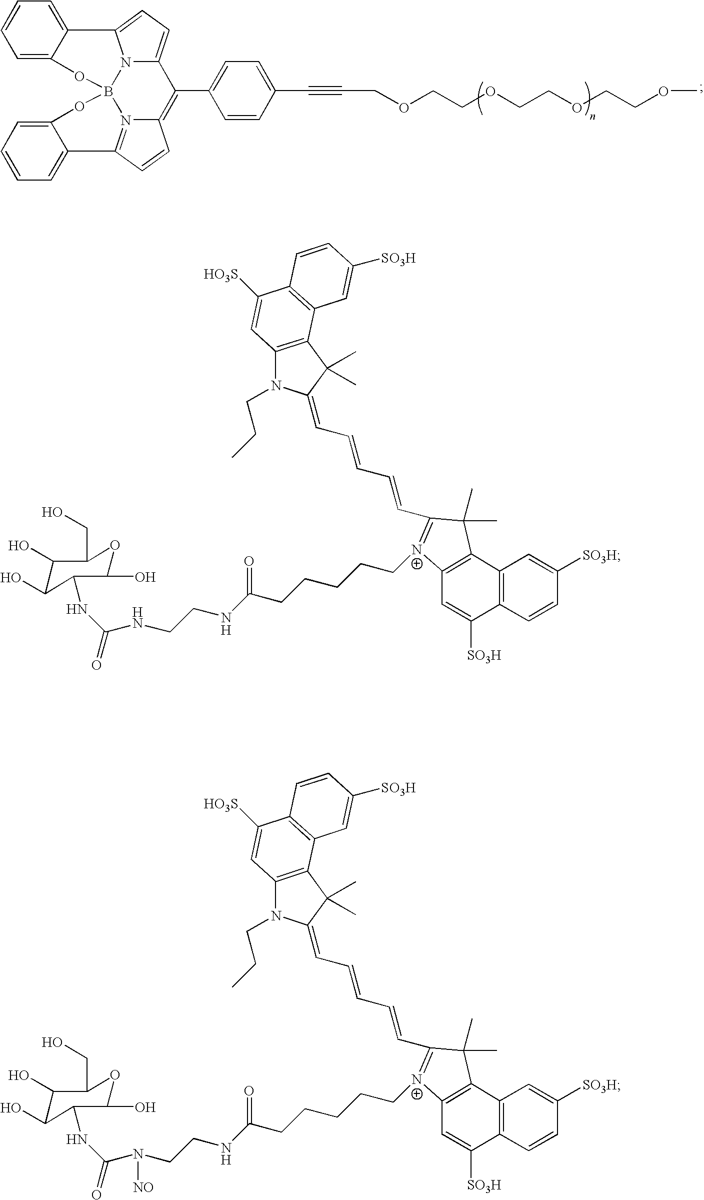

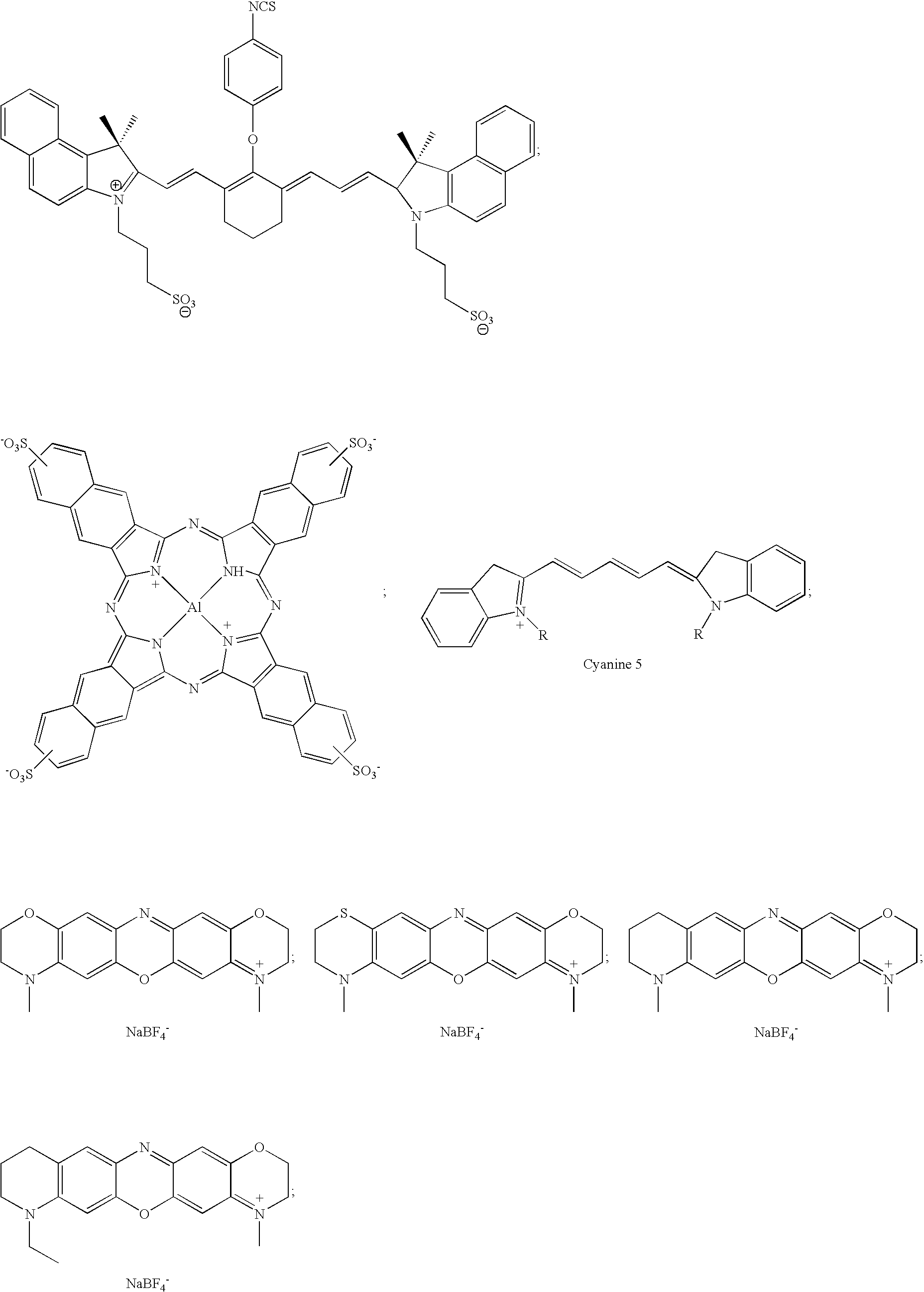

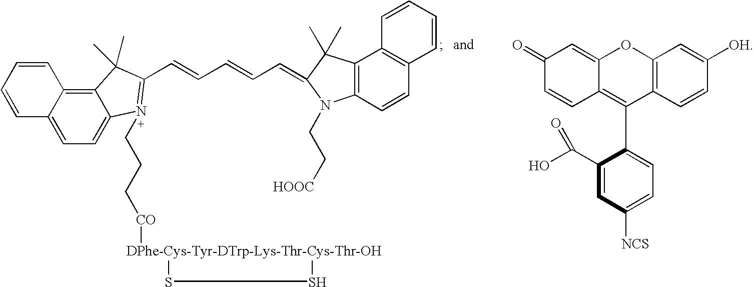

These near infrared imaging groups are disclosed in, for example, Tetrahedron Letters 49 (2008) 3395-3399; Angew. Chem. Int. Ed. 2007, 46, 8998-9001; Anal. Chem. 2000, 72, 5907; Nature Biotechnology vol 23, 577-583; Eur Radiol(2003) 13: 195-208; and Cancer 67: 1991 2529-2537, which are herein incorporated by reference in their entirety.

The near infrared imaging group can be coupled to at least one benzothiazole group, benzene group, or be an R₁-R₁₃ group. In one example, the near infrared imaging group can be coupled to at least one benzene group.

In one example, the molecular probe with the near infrared imaging group can have the following formula:

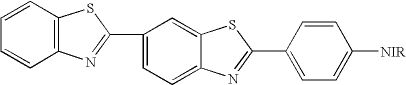

wherein NIR is a near infrared imaging group; or a salt thereof.

By way of example, the molecular probe can include a compounds having the following formula:

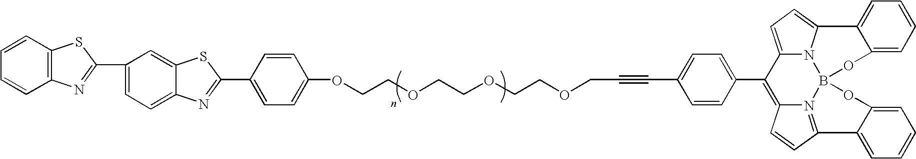

wherein n is 3 to 10; or a salt thereof.

The foregoing formulae represent the general structures of compounds found to be effective molecular probes for labeling amyloid in vivo as well as in vitro as described in the examples below. The molecular probes are characterized by their ability to enter the brain and selectively localize in amyloid deposits with high affinity. In order to facilitate the delivery of the molecular probes of the present invention to amyloid deposits in the brain and elsewhere in a subject, the molecular probes may be combined with a pharmaceutically acceptable carrier or excipient.

When referring to a compound of the present invention, it is intended that the term “compound” encompass not only the specified molecular entity but also pharmaceutically acceptable formulations, including, but not limited to salts, esters, amides, conjugates, active metabolites, and other such derivatives, analogs, and related structures.

As used herein, the term “pharmaceutically acceptable” is meant as a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration Center for Drug Evaluation and Research.

In certain embodiments of the present invention, the molecular probes described above can be used to detect amyloid in a subject in vivo or from a tissue sample in vitro. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common

X-ray diffraction and infrared spectra. Amyloids are composed of a proteinaceous fibrillar material and are found deposited in various tissues and organs, sometimes secondary to a chronic inflammatory disease.

An example of an amyloid which may be detected by the molecular probes of the present invention is Aβ, also known as β-amyloid peptide, amyloid beta, or A4 peptide (see U.S. Pat. No. 4,666,829; Glenner & Wong, Biochem. Biophys. Res. Commun. 120, 1131 (1984)). Aβ is a peptide of 39-43 amino acids, which is the principal component of characteristic plaques of Alzheimer's disease. Aβ is generated from the metabolic processing of a larger protein APP by two enzymes, termed β and γ secretases, in the endoplasmic reticulum (“ER”), the Golgi apparatus, or the endosomal-lysosomal pathway (Selkoe(1994), Annu. Rev. Cell Biol. 10:373-403).

The molecular probes used in the claimed methods can be used to label any of the naturally occurring forms of Aβ peptide, and particularly the human forms (i.e., Aβ39, Aβ40, Aβ41, Aβ42 or Aβ43) (see Hardy et al. (1997) TINS 20:155-158). Aβ41, Aβ40 and Aβ39 differ from Aβ42 by the omission of Ala, Ala-Ile, and Ala-Ile-Val respectively from the C-terminal end. Aβ43 differs from Aβ42 by the presence of a threonine residue at the C-terminus. Although Aβ40 is the predominant form produced in humans, 5-7% of total Aβ exists as Aβ42 (Cappai et al. (1999), Int. J. Biochem. Cell Biol. 31:885-89).

The molecular probes may also localize to an active fragment or analog of a natural Aβ peptide. Analogs include allelic, species and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N or C terminal amino acids. Examples of unnatural amino acids are α,α-disubstituted amino acids, N-alkyl amino acids, lactic acid, 4-hydroxyproline, γ-carboxyglutamate, ε-N,N,N-trimethyllysine, ε-N-acetyllysine, O-phosphoserine, N-acetylserine, N-formylmethionine, 3-methylhistidine,

5-hydroxylysine, ω-N-methylarginine.

In certain embodiments, the molecular probes of the present invention can be administered to an animal and utilized for labeling and detecting in vivo amyloid deposits in the animal's brain tissue. Amyloid deposits, which can be imaged in an animal's brain using the molecular probes of the present invention, are typically found in the forms of senile plaques (SPs) and neurofibrillary tangles (NFTs).

SPs are extracellular deposits of amyloid in the gray matter of the brain of humans and other animals (e.g., mammals and birds). The SP deposits are associated with degenerative neural structures and an abundance of microglia and astrocytes. The plaques are variable in shape and size, but are on the average 50 μm in size. In Alzheimer's disease, they are primarily composed of Aβ peptides. These polypeptides tend to aggregate and are believed to be neurotoxic.

Neurofibrillary tangles are an intracellular abnormality, involving the cytoplasm of nerve cells. Neurofibrillary tangles were first described by Alois Alzheimer in one of his patients suffering from Alzheimer's disease. Neurofibrillary tangles are composed mainly of abnormally phosphorylated tau protein (a neuron-specific phosphoprotein that is the major constituent of neuronal microtubules). Variable amounts of other proteins can also be found attached to the abnormally-phosphorylated tau protein of the neurofibrillary tangles. Neurofibrillary tangles in pyramidal neurons of the cerebral cortex often have a flame-shape appearance, filling the neuronal cell body and apical dendrite. In other neurons, neurofibrillary tangles often have a more spherical (globose or globoid) appearance. In the neuropil of the cerebral cortex, short, sometimes curly, threadlike structures (termed neuropil threads or dystrophic neurites) represent neuronal dendrites or axons containing the neurofibrillary tangles.

Neurofibrillary tangles can be detected in a variety of other neurologic disorders: in substantia nigra neurons in postencephalitic Parkinsonism, throughout the nervous system in the Parkinsonism-dementia-ALS complex disorder of the Chamorro population on Guam, in the cerebral cortex in dementia pugilistica (“punch-drunk syndrome”), and in the brain stem and thalamus in Steele-Richardson-Olszewski progressive supranuclear palsy (PSP). It is also believed that neurofibrillary tangles are seen in Creutzfeldt-Jakob disease.

For the purposes of the present invention, the molecular probes can be administered to an animal's brain tissue, where the animal's brain tissue is typically a mammal's brain tissue, such as a primate, e.g., chimpanzee or human; cow; dog; cat; a rodent, e.g. guinea pig, rat, mouse; rabbit; or a bird; reptile; or fish. The molecular probes can be administered to an animal parenterally, typically through intravenous injection. “Administered”, as used herein, means provision or delivery of a molecular probe in an amount(s) and for a period of time(s) effective to label amyloid in a subject.

The molecular probes of the present invention can be used for neuroanatomical or neuropathological studies. For example, researchers studying normal brains can employ the methods described herein to examine the morphology and distribution of amyloid in an animal.

“Distribution” as used herein is the spatial property of being scattered about over an area or volume. In this case, the “the distribution of amyloid” is the spatial property of amyloid being scattered about over an area or volume included in the animal's brain tissue. One skilled in the art may use the molecular probes of the present invention to assess the amyloid distribution in a subject's brain and correlate the distribution to a specific disorder or disease state. In addition, one may also utilize the molecular probes to quantify the amyloid load in a subject.

The molecular probes of the present invention can also be used to detect a neurodegenerative disorder in an animal through the use of in vivo amyloid labeling. Thus, in certain embodiments, the molecular probes described herein can be administered to an animal. The distribution of the molecular probe in the animal's brain tissue can then be visualized (e.g., with an in vivo imaging modality). The distribution of the molecular probe may then be correlated with the presence or absence of a neurodegenerative disorder. A distribution may be dispositive for the presence or absence of a neurodegenerative disorder or may be combined with other factors and symptoms by one skilled in the art to positively detect the presence or absence of a neurodegenerative disorder.

In one example of detecting a neurodegenerative disorder, the methods described herein can be used to compare amyloid deposits in normal brain tissues of control populations to those of a suspect animal. If the suspect animal has a neurodegenerative disorder, the amyloid load may be higher in the suspect animal compared to a control, thus possibly indicating the presence of a neurodegenerative disorder, subject to the interpretation of one skilled in the art. “Control” or “Control Population” as used herein are defined as a group of individual animals (or samples thereof) not having a neurodegenerative disorder.

More specifically, the molecular probes and methods provided of the present invention can be used to detect an amyloid related disorder in an animal through the use of in vivo amyloid labeling. “Amyloidosis”, “amyloid disease” or “amyloid-related disease” refers to a pathological condition characterized by the presence of amyloid fibers. Amyloid-related diseases can either be restricted to one organ or spread to several organs.

Some amyloid diseases can be idiopathic, but most appear as a complication of a previously existing disorder. For example, primary amyloidosis (AL amyloid) can appear without any other pathology or can follow plasma cell dyscrasia or multiple myeloma. There are many forms of hereditary systemic amyloidoses. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins.

Primary amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia. Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the kidney, liver, spleen and heart, may be involved.

Secondary amyloidosis is usually seen associated with chronic infection (such as tuberculosis) or chronic inflammation (such as rheumatoid arthritis). A familial form of secondary amyloidosis is also seen in other types of familial amyloidosis, e.g., Familial Mediterranean Fever (FMF). This familial type of amyloidosis is genetically inherited and is found in specific population groups. In both primary and secondary amyloidosis, deposits are found in several organs and are thus considered secondary amyloid diseases. Localized amyloidosis tends to involve a single organ system. Deposition of secondary amyloidosis fibrils can be widespread in the body, with a preference for parenchymal organs. The kidneys are usually a deposition site, and the liver and the spleen may also be affected. Deposition is also seen in the heart, gastrointestinal tract, and the skin.

Underlying diseases, which can lead to the development of secondary amyloidosis include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. Secondary amyloidosis deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in secondary amyloidosis fibril amyloid deposits. These include such conditions as Hodgkin's lymphoma, renal carcinoma, carcinomas of gut, lung and urogenital tract, basal cell carcinoma, and hairy cell leukemia. Other underlying conditions that may be associated with secondary amyloidosis are Castleman's disease and Schnitzler's syndrome.

Different amyloids are further characterized by the type of protein present in the deposit. For example, neurodegenerative diseases such as scrapie, bovine spongiform encephalitis, Creutzfeldt-Jakob disease, and the like are characterized by the appearance and accumulation of a protease-resistant form of a prion protein (referred to as AScr or PrP-27) in the central nervous system. Similarly, Alzheimer's disease is characterized by neuritic plaques and neurofibrillary tangles. In this case, the amyloid plaques found in the parenchyma and the blood vessel is formed by the deposition of fibrillar Aβ protein. Other diseases such as adult-onset diabetes (type II diabetes) are characterized by the localized accumulation of amyloid fibrils in the pancreas.

In another type of amyloidosis seen in patients with type II diabetes, the amyloidogenic protein IAPP, when organized in oligomeric forms or in fibrils, has been shown to induce β-islet cell toxicity in vitro. Hence, appearance of IAPP fibrils in the pancreas of type II diabetic patients contributes to the loss of the β islet cells (Langerhans) and organ dysfunction which can lead to insulinemia.

Another type of amyloidosis is related to β₂ microglobulin and is found in long-term hemodialysis patients. Patients undergoing long term hemodialysis will develop β₂-microglobulin fibrils in the carpal tunnel and in the collagen rich tissues in several joints. This causes severe pains, joint stiffness and swelling. β₂ microglobulin is a 11.8 kiloDalton polypeptide and is the light chain of Class I MHC antigens, which are present on all nucleated cells. Under normal circumstances, β₂M is usually distributed in the extracellular space unless there is an impaired renal function, in which case β₂M is transported into tissues where it polymerizes to form amyloid fibrils. Failure of clearance such as in the case of impaired renal function, leads to deposition in the carpal tunnel and other sites (primarily in collagen-rich tissues of the joints). Unlike other fibril proteins, β₂M molecules are not produced by cleavage of a longer precursor protein and are generally present in unfragmented form in the fibrils. Retention and accumulation of this amyloid precursor has been shown to be the main pathogenic process underlying DRA. DRA is characterized by peripheral joint osteoarthropathy (e.g., joint stiffness, pain, swelling, etc.). Isoforms of β₂M, glycated β₂M, or polymers of β₂M in tissue are the most amyloidogenic form (as opposed to native β₂M). Unlike other types of amyloidosis, β₂M is confined largely to osteoarticular sites. Visceral depositions are rare. Occasionally, these deposits may involve blood vessels and other important anatomic sites.

Another type of amyloidosis is cerebral amyloid angiopathy (CAA). CAA is the specific deposition of amyloid-β fibrils in the walls of leptomingeal and cortical arteries, arterioles and veins. It is commonly associated with Alzheimer's disease, Down's syndrome and normal aging, as well as with a variety of familial conditions related to stroke or dementia (see Frangione et al.(2001), Amyloid: J. Protein Folding Disord. 8(Suppl. 1):36-42).

In another embodiment, the methods included in the present invention can be used to detect mild cognitive impairment. Mild Cognitive Impairment (“MCI”) is a condition characterized by a state of mild but measurable impairment in thinking skills, which is not necessarily associated with the presence of dementia. MCI frequently, but not necessarily, precedes Alzheimer's disease.

It has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson et al. (2002), Proc. Natl. Acad. Sci. USA 99(18): 11830-5).

Additionally, abnormal accumulation of APP and of amyloid-β protein in muscle fibers has been implicated in the pathology of sporadic inclusion body myositis (IBM) (Askanas, V., et al. (1996) Proc. Natl. Acad. Sci. USA 93:1314-1319). Accordingly, the molecular probes of the invention can be used to detect disorders in which amyloid-beta protein is abnormally deposited at non-neurological locations, such as treatment of IBM by delivery of the compounds to muscle fibers.

A specific example of an amyloid related disorder in which the molecular probes of the present invention can be used to detect is Alzheimer's disease. The presence of amyloid containing senile plaques and neurofibrillary tangles are known to be an important criterion of the neuropathological-histological diagnosis of neurodegenerative disorders such as Alzheimer's disease. The principal constituent of the senile plaques is Aβ. Aβ, as described above, is a peptide with an internal fragment of 39-43 amino acids of a precursor protein termed amyloid precursor protein (APP). In Alzheimer's disease, neurofibrillary tangles are generally found in the neurons of the cerebral cortex and are most common in the temporal lobe structures, such as the hippocampus and amygdala.

The formation and the distribution of the pathological neurofibrillaries have a regularity and allows one skilled in the art to not only diagnose a neurodegenerative disorder, such as Alzheimer's disease, but to also stage the disease (Braak et al. (1993) European Neurology 33: 403-408). Other factors which may be measured in conjunction with the presence of amyloid in the diagnosis of Alzheimer's include dementia, atrophic brain with hydrocephalus, and other degenerative signs. These factors in combination with the occurrence of a great number of plaques allows on skilled in the art to diagnose Alzheimer's disease with high probability. The molecular probes of the present invention may be particularly useful in animal models of Alzheimer's disease (see McGowan et al. (2006) Trends in Genet. 22(5):281-289 for review of mouse models of Alzheimer disease).

Additional neurodegenerative disorders, in which the methods described herein may detect, can include any disease, condition, or disorder related to neurodegeneration in an animal. A neurodegenerative disorder as used herein can arise from stroke, heat stress, head and spinal cord trauma (blunt or infectious pathology), and bleeding that occurs in the brain. Examples of neurodegenerative disorders include Alexander disease, Alper's disease, Amyotrophic lateral sclerosis, Ataxia telangiectasia, Spielmeyer-Vogt-Sjogren-Batten disease, Bovine spongiform encephalopathy, Canavan disease, Cockayne syndrome, Corticobasal degeneration, Creutzfeldt-Jakob disease, Huntington Disease, HIV-associated dementia, Kennedy's disease, Krabbe disease, Lewy body dementia, Spinocerebellar ataxias, Multiple Sclerosis, Multiple system atrophy, Neuroborreliosis, Parkinson's disease, Pelizaeus-Merzbacher disease, Pick's disease, Primary lateral sclerosis, Prion diseases, Refsum's disease, Sandhoff disease, Schilder's disease, Spinal muscular atrophy, Steele-Richardson-Olszewski disease, and tabes dorsalis.

In another aspect of the present invention, a method of quantifying the amyloid load in an animal is provided. The method includes first administering in vivo to the animal a molecular probe as described herein. The distribution of the molecular probe may then be visualized in the animal's brain (e.g. with an in vivo imaging modality). For directly monitoring amyloid deposit changes in the brain of an animal, embodiments of the invention can readily penetrate the blood-brain barrier (BBB) and localize to amyloid in proportion to the amyloid load in a subject. Radiolabeled molecular probes of the present invention can be used as imaging markers in conjunction with an in vivo imaging modality to directly assess the extent of the amyloid load in an animal. Finally, the distribution of the molecular probe may be correlated by one skilled in the art with the amyloid load in the animal.

The deposition of cerebral amyloid plaques is a hallmark feature of Alzheimer's disease (AD) and a reduction of amyloid load is widely regarded as the most promising therapy for the disease. The ability to track and/or quantify the amyloid load in a subject may provide a useful tool to researchers and clinicians. Amyloid load or amyloid burden, as used herein, is the amount of amyloid plaque in a given animal or tissue sample. A reduction in amyloid load, as used herein, is the inhibition and/or dissolution of amyloid plaque formation in a subject.

The methods provided can be used to monitor and compare the amyloid load in an animal prior to a given therapy, during a given therapy, or post therapeutic regimen. A reduction in an amyloid load in an animal may be indicative of the efficacy of a given therapy. This can provide a direct clinical efficacy endpoint measure of anti-amyloid therapies. Therefore, in another aspect of the present invention, a method of monitoring the efficacy of a neurodegenerative disorder therapy is provided. More specifically the present invention provides for a method of monitoring the efficacy of an anti-amyloid therapy. The term therapy, as used herein, includes the administration or application of remedies to an animal for a neurodegenerative disorder or injury; medicinal or surgical management; treatment.

The methods of monitoring the efficacy of a neurodegenerative disorder include the steps of administering in vivo to the animal a molecular probe as described herein, then visualizing a distribution of the molecular probe in the animal (e.g., with an in vivo imaging modality as described herein), and then correlating the distribution of the molecular probe with the efficacy of the anti-amyloid therapy. It is contemplated that the administering step can occur before, during, and after the course of a therapeutic regimen in order to determine the efficacy of a chosen therapeutic regimen. One way to assess the efficacy of the anti-amyloid therapy is to compare the distribution of a molecular probe pre and post anti-amyloid therapy.

An efficacious therapy or the efficacy of a given therapy, for example, may be any therapy directed to reduce amyloid load which results in an increase in neuronal survival. An efficacious therapy may act to ameliorate the course of an amyloid related disease using any of the following mechanisms, such as, for example but not limited to: slowing the rate of amyloid fibril formation or deposition; lessening the degree of amyloid deposition; inhibiting, reducing, or preventing amyloid fibril formation; inhibiting amyloid induced inflammation; enhancing the clearance of amyloid from, for example, the brain; or protecting cells from amyloid induced (oligomers or fibrillar) toxicity. For example, an anti-amyloid therapy can include administration of a therapeutic agent or therapies aimed at the endogenous reduction of amyloid or amyloid deposits in an animal.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

### Example 1

We studied a series of dibenzothiazole derivatives as amyloid-imaging agents. The dibenzothiazole pharmacophore is seen in several histological dyes such as primuline (Klut et al. (1989) Histochem. J. 21:645-650) and thioflavin S (ThS) (Churukian et al. (2000) Biotech. Histochem. 75:147-150, Westermark et al. (1999) Methods Enzymol. 309:3-25, Bancroft and Gamble (2002) Theory and Practice of Histological Techniques, 5^{th} ed.). Primuline and ThS have been commonly used as viability stains of starch in phytoplankton and a fluorescent stain for amyloid, respectively. However, Primuline and ThS exist as a mixture of several components. The major component of these dyes contains two conjugated benzothiazole units (Colour Index 3^{rd} ed. (1971) Society of Dyers and Colourists). We thus designed and synthesized a series of dibenzothiazole derivatives. Compared with primuline and ThS, these dibenzothiazole derivatives are lipophilic and readily enter the brain, making it possible for potential in vivo amyloid-imaging agents. In vitro evaluations suggested that these dibenzothiazole derivatives bound to amyloid deposits in AD brain homogenates with high affinities. In vivo brain permeability studies of selected compounds displayed high initial brain uptake. These studies thus expand the current portfolio of amyloid-imaging agents for potential clinical applications.

All chemicals were purchased from Sigma-Aldrich and used without further purification. ¹H NMR spectra were obtained at 300 MHz on Bruker DPX-300 (QNP probe) NMR spectrometers using 5 mm NMR tubes (Wilmad 528-PP) in CDCl₃ or DMSO-d₆ (Aldrich or Cambridge Isotopes) solutions at room temperature. Chemical shifts are reported as δ values relative to internal TMS. HR-ESIMS were acquired under the electron spray ionization (ESI) condition. The radioactivities of ¹²⁵I and ³H were calculated by the counts per minute in a c counter (Cobra Packard model U5005) and a multiple-purpose scintillation counter (Beckman, LS 6500). Radiochemical purity was determined by Hewlett Parkard high-pressure liquid chromatography (HPLC) system equipped with UV and Bioscan flow count detectors.

### Chemistry, Synthesis, and Radiolabeling

The synthesis of dibenzothiazole derivatives is described in Scheme 1 starting from commercially available paminobenzothiazole (1). As shown in Scheme 1, 2-aminobenzothiazole-6-carboxylic acid (2) was first prepared from 1 based on previously reported procedures (Schubert et al. (1947) Justus Liebigs Ann. Chem. 558:10). Basic hydrolysis of 2 followed by neutralizing in HCl and ZnCl₂ yielded the Zinc salt of 4-amino-3-mercaptobenzoicacid (3), which was coupled immediately with p-nitrobenzoyl chloride to give 2-(4-nitro-phenyl)-benzothiazole-6-carbolic acid (4) with 83% yield. The 6-carbolic acid of 4 was then converted into acyl chloride (5) followed by coupling with a 5-substituted aminothiophenol to give 6″-substitute-2′-(4-nitro-phenyl)-[2,6′]dibenzothiazolyl (6-9). Reduction of 6-9 with SnCl₂ in ethanol afforded 6″-substitute-2′-([2,6′]dibenzothiazolyl-2′-yl)-aniline (10-13), which can be further methylated with methyliodide and K₂CO₃ in DMSO to monomethylamino derivatives (14-16) and dimethylamino derivatives (17).

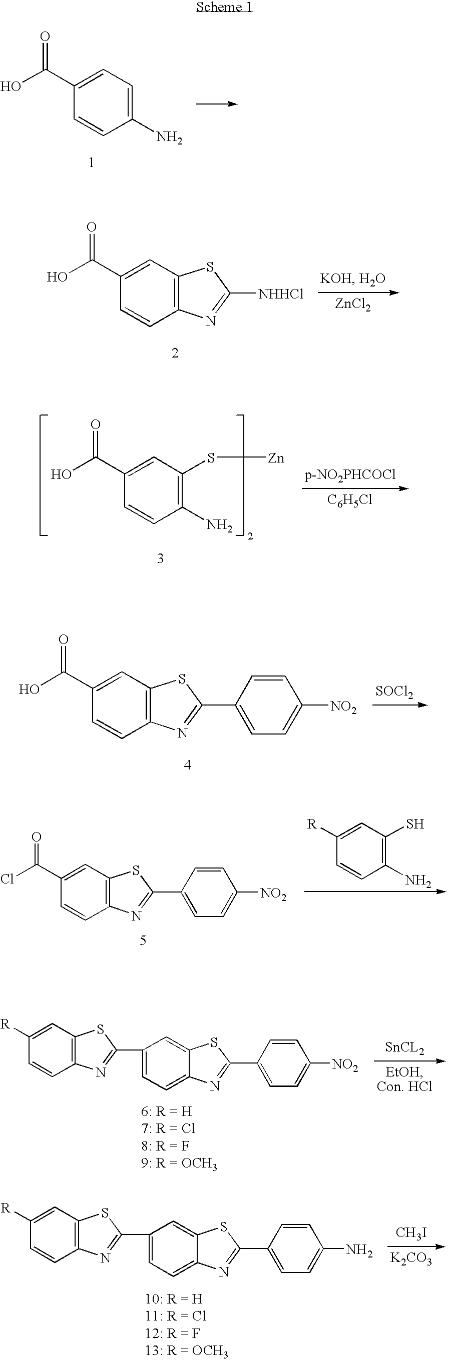

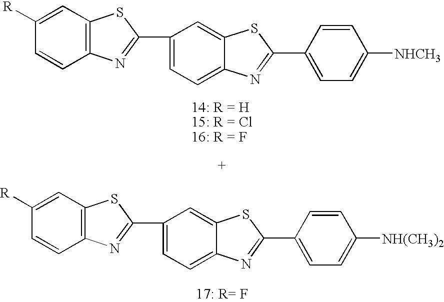

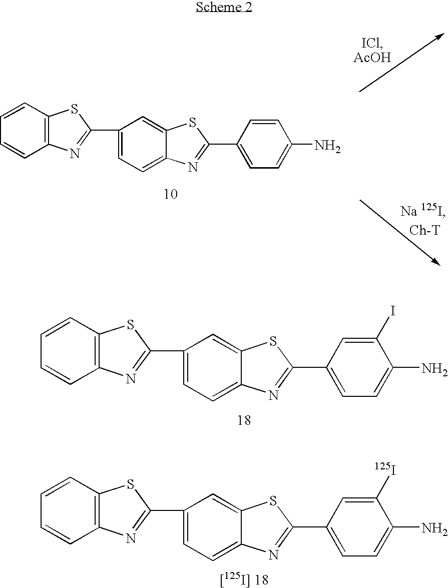

### Synthesis of 2-aminobenzothiazole-6-carbolic Acid(2)

NaSCN (65 g, 0.8 mol) was added to a suspension of commercially available 4-amino-benzoic acid (1, 100 g, 0.73 mol) in MeOH followed by the addition of Br₂ (38 ml, 0.73 mol) in portions. The above solution was allowed to cool to −10° C. and stirred for 2 h while keeping the inner temperature below −5° C. The precipitate was then filtered and suspended in 350 ml of 1 M HCl. The suspension was heated to reflux for 30 min. After immediate filtration, 150 ml concd HCl was added to the hot filtrate to give 70 g (yield 42%) of 2-amino-benzothiazole-6-carboxylic acid (2) (as a white solid), which was dried and used without further purification.

### Synthesis of Zinc Salt of 4-amino-3-mercaptobenzoic Acid(3)

Under Argon, compound 2 (9.18 g, 40 mmol) was dissolved in a KOH solution (45 g KOH/45 ml water) and heated to reflux for 3 h. After being cooled to room temperature, the solution was neutralized by concd HCl (50 ml). Then ZnCl2 in 25 ml of water was added slowly while white solid precipitated out. The suspension was acidified by AcOH. The solid was filtered, washed with water, and dried in a vacuum to give 8.18 g (98%) of 4-amino-3-mercaptobenzoic acid (3) as a white solid. ¹H NMR (300 MHz, DMSO-d₆) δ12.02 (br, 2H), 7.90 (s, 1H), 7.63 (d, J=7.0 Hz, 1H), 7.53 (s, 1H), 7.30 (d, J=8.0 Hz, 1H), 6.74 (d, J=8.5 Hz, 1H), 6.55 (d, J=8.1 Hz, 1H), 6.24 (br, 2H), 5.66 (br, 2H).

### Synthesis of 2-(40-nitrophenyl)-6-(benzothiazolyl)benzothiazole(4)

Compound 3 (8.18 g, 20 mmol) was suspended in pyridine (50 ml) and heated to 80° C. p-Nitrobenzoyl chloride (7.95 g, 42.8 mmol) was added in portions to give a clear solution, which was stirred for another hour. After being cooled to room temperature, the precipitate was filtered, washed with dilute hydrochloric acid and water, and dried under a vacuum to afford 9.95 g (83%) of 2-(4′-nitrophenyl)-6-(benzothiazolyl)benzothiazole (4), which was used directly without further purification.

### Synthesis of 2-(4-nitro-phenyl)-benzothiazole-6-carbonyl Chloride(5)

Compound 4 (1.00 g, 3.3 mmol) was suspended in SOCl₂ (5 ml) and heated to reflux for 1 h. Then excess SOCl₂ was evaporated under reduced pressure to get 2-(4-nitro-phenyl)-benzothiazole-6-carbonyl chloride (5), which was used without further purification.

### General Synthesis of 600-substitute-20-(4-nitro-phenyl)-[2,60]dibenzothiazolyl(6-9)

To a suspension of 5 in chlorobenzene (28 ml), 5-substituted aminothiophenol (2-aminothiaphenol (0.60 g, 4.8 mmol), 2-amino-5-chloro-benzenethiol (0.60 g, 3.75 mmol),

2-amino-5-fluoro-benzenethiol (0.60 g, 4.2 mmol), and 2-amino-5-methoxy-benzenethiol (0.65 g, 4.19 mmol)) were added, respectively. The obtained mixtures were heated to reflux for 3 h. After being cooled to room temperature, the solids were filtered and dried under vacuum to give 6 (1.00 g, 79%), 7 (1.00 g, 73%), 8 (1.03 g, 76%), and 9 (1.02 g, 74%).

### General Synthesis of 4-(6-substitute-[2,60]dibenzothiazolyl-20-yl)-phenylamine(10-13)

To a suspension of 6-9 in concd HCl (13 ml), ethanol (100 ml), and SnCl₂ (2.00 g, 10.0 mmol) were added. The suspension was heated to 80° C. for 1 h. After being cooled to room temperature; the solid was filtered; washed with concentrated HCl, water, and dilute ammonium; and dried in vacuum to give 10 (0.88 g, quant.), ¹H NMR (300 MHz, DMSO-d₆) δ 8.82 (d, J=1,5 Hz, 1H), 8.18 (d, J=8.5 Hz, 2H), 8.08 (d, J=8.0 Hz, 1H), 8.03 (d, J=8.5 Hz, 1H), 7.82 (d, J=8.5 Hz, 2H), 7.57 (t, J=7.3 Hz, 1H), 7.48 (t, J=7.3 Hz, 1H), 6.69 (d, J=8.5 Hz, 2H), 6.03 (s, 2H). HR-ESIMS: m/z calcd for C₂₀H₁₄N₃S₂ (M+H⁺): 360.0629, found 360.0631. Compound 11 (0.9 g, quant.), ¹H NMR (300 MHz, DMSO-d₆) δ 8.82 (s, 1H), 8.35 (s, 1H), 8.18 (d, J=4.4 Hz, 2H), 8.06 (t, J=8.8 Hz, 2H), 7.81 (d, J=8.5 Hz, 2H), 7.60 (d, J=4.3 Hz, 1H), 6.71 (d, J=8.5 Hz, 2H), 6.04 (s, 1H). HR-ESIMS: m/z calcd for C₂₀H₁₂ClN₃S₂ (M+H⁺): 394.0239, found 394.0225. Compound 12 (0.83 g, quant.), ¹H NMR (400 MHz, DMSO-d₆) δ 8.80 (d, J=6.3 Hz, 1H), 8.01-8.18 (m, 6H), 7.83 (d, J=7.4 Hz, 2H), 7.44 (d, J=4.6 Hz, 1H), 6.71 (d, J=7.7 Hz, 2H). HR-ESIMS: m/z calcd for C₂₀H₁₂FN₃S₂ (M+H⁺): 378.0535, found 378.0523. Compound 13 (1.03 g, quant), ¹H NMR (400 MHz, DMSO-d₆) δ 8.43 (d, J=2.9 Hz, 1H), 7.98 (dd, J=6.9, 8.7 Hz, 2H), 7.80 (t, J=8.6 Hz, 2H), 7.14 (m, 3H), 6.69 (d, J=8.6 Hz, 2H), 6.02 (s, 2H), 3.87 (s, 3H). HR-ESIMS: m/z calcd for C₂₁H₁₅N₃OS₂ (M+H⁺): 390.0735, found 390.0728.

### General Synthesis of [4-(6-substitute-[2,60]dibenzothiazolyl-20-yl)-phenyl]-methylamine(14-15)

Under Argon, compounds 10-11 (1.30 mmol) and K₂CO₃ (1.15 g, 8.34 mmol) were suspended in DMSO (15 ml) followed by an addition of MeI (0.17 ml, 2.78 mmol). The sealed vial was heated to 100° C. and stirred for 31 h. The solution was diluted with ethyl acetate and washed with water and brine, and dried over Na₂SO₄. After evaporating the solvent, the crude product was purified by flash column chromatography (hexane/ethyl acetate=4:1-2:1) to give 14 (36 mg, 7%). ¹H NMR (300 MHz, DMSO-d₆) δ8.83 (s, 1H), 8.18 (d, J=8.0 Hz, 2H), 8.08 (d, J=8.0 Hz, 1H), 8.04 (d, J=8.5 Hz, 1H), 7.88 (d, J=8.6 Hz, 1H), 7.55 (t, J=7.0 Hz, 1H), 7.46 (t, J=7.0 Hz, 1H), 6.69 (d, J=8.6 Hz, 2H), 6.62 (d, J=5.1 Hz, 1H), 2.78 (d, J=5.1 Hz, 3H), HR-ESIMS: m/z calcd for C₂₁H₁₆N₃S₂ (M+H⁺): 374.0786, found 374.0797. Compound 15 (100 mg, 19%). ¹H NMR (300 MHz, DMSO-d₆) δ8.82 (s, 1H), 8.35 (s, 1H), 8.18 (d, J=4.3 Hz, 1H), 8.05 (t, J=8.7 Hz, 2H), 7.81 (d, J=8.7 Hz, 2H), 7.61 (d, J=4.4 Hz, 1H), 6.68 (d, J=8.8 Hz, 2H), 6.63 (s, 1H), 2.79 (s, 3H). HR-ESIMS: m/z calcd for C₂₁H₁₄ClN₃S₂ (M+H⁺): 408.0396, found 408.0382.

### Synthesis of [4-(6-fluoro-[2,60]dibenzothiazolyl-20-yl)-phenyl]-methylamine(16) and [4-(6-fluoro-[2,60]dibenzothiazolyl-20-yl)-phenyl]-dimethylamine(17)

Under Argon, the compound 12 (0.50 g, 1.32 mmol) and K₂CO₃ (1.10 g, 6 mmol) were suspended in DMSO (15 ml) and MeI (0.17 ml, 2.78 mmol) was added. The sealed vial was heated to 100° C. and stirred for 31 h. The solution was diluted with ethyl acetate and washed with water and brine, dried on Na₂SO₄, concentrated, and purified by column chromatography (hexane/ethyl acetate=4:1-2:1) to give compound 16 (58 mg, 22%) and compound 17 (91 mg, 34%). Compound 16: ¹H NMR (400 MHz, DMSO-d₆) δ8.38 (s, 1H), 7.95-8.21 (m, 4H), 7.87 (d, J=7.3 Hz, 2H), 7.44 (d, J=4.5 Hz, 1H), 6.69 (d, J=7.6 Hz, 2H), 2.38 (s, 3H). HR-ESIMS: m/z calcd for C₂₁H₁₄FN₃S₂ (M+H⁺): 392.0691, found 392.0680. Compound 17: ¹H NMR (400 MHz, DMSO-d₆) δ8.42 (s, 1H), 8.00-8.20 (m, 4H), 7.79

(d, J=7.3 Hz, 2H), 7.45 (d, J=4.5 Hz, 1H), 6.70 (d, J=7.5 Hz, 2H), 2.44 (s, 6H). HR-ESIMS: m/z calcd for C₂₂H₁₆FN₃S₂ (M+H⁺): 406.0848, found 406.0844.

### Synthesis of 4-[2,60]dibenzothiazolyl-20-yl-2-iodophenylamine(18)

Under Argon, ICl (0.07 ml) was added dropwise to the suspension of 10 (20 mg, 0.056 mM) in AcOH (10 ml). The resulting mixture was sealed and stirred at room temperature for 18 h. The reaction was quenched with ethanol and the solvent was removed. The residue was purified by preparative TLC to get 4-[2,6′]dibenzothiazolyl-2′-yl-2-iodo-phenylamine (18, 10 mg, 38%) as brown solid. ¹H NMR (300 MHz, DMSO-d₆) δ8.87 (s, 1H), 8.44 (s, 1H), 8.40 (d, J=3.6 Hz, 1H), 8.32 (d, J=1.8 Hz, 1H), 7.82 (d, J=8.5 Hz, 2H), 7.64 (s, 1H), 7.57

(t, J=7.3 Hz, 1H), 7.48 (t, J=7.3 Hz, 2H), 6.69 (d, J=8.5 Hz, 1H), 6.10 (s, 1H). HR-ESIMS: m/z calcd for C₂₀H₁₂IN₃S₂ (M+H⁺): 485.9596, found 485.9588.

### Synthesis of 4-[2,6′]dibenzothiazolyl-2′-yl-2-¹²⁵I-phenylamine([¹²⁵I]18)

To a solution of 10 (1 mg) in 1 ml acetic acid was added sodium [¹²⁵I]iodide (specific activity 83.05 TBq/mmol) in 0.01 M sodium hydroxide solution. Following the addition of 50 μl Chloramine T solution (ChT, 30 mg dissolved in 500 μl acetic acid), the reaction mixture was stirred at room temperature for 3 h, and quenched with 200 μ/L sodium hydrogensulfite (1 M) solution. The mixture was diluted with 20 ml of water and adjusted to pH 7-8 with saturated NaHCO₃. The reaction mixture was then loaded onto a Waters C-8 Sep-Pak™ plus cartridge. The Sep-Pak cartridge was washed with 10 ml of water and dried with a rapid air bolus, and the radioiodinated product was slowly eluted with 2 ml of methanol. The solution was concentrated under nitrogen to about 200 μl and the crude product was purified by HPLC using a Phenomenex C-18 column (250×4.6 mm, acetonitrile:TEA buffer (pH 7.5)=85:15, flow rate 1.0 ml/min, ^{t}R=17.31 min). The desired fractions were collected, diluted with 50 ml of water, and loaded onto a water C-8 Sep-Pak™ plus cartridge. After being washed with another 10 ml of water and dried with a rapid air bolus, the cartridge was eluted with 2 ml ethanol and dried under N₂ to give the final product [¹²⁵I] 18 in overall 20-30% radiochemical yields with radiochemical purities of >98% after purification by HPLC.

### Partition Coefficient Determination

Partition coefficients were measured by mixing [¹²⁵I] 18 (10 μl, RCP>98%, approximately 50,0000 cpm) with sodium phosphate buffer (PBS, 3 g, 0.1 M, pH 7.4) and

n-octanol (3 g, 3.65 ml) in test tubes. The tubes were vortexed for 3 min (1 min 3×) at room temperature followed by centrifugation at 3500 rpm for 5 min. Then 1 ml of buffer and 1 ml of n-octanol were taken out, weighed, and counted. The partition coefficient was determined by calculating the ratio of cpm/g of n-octanol to that of PBS and expressed as logP oct=log [cpm/g (n-octanol)/cpm/g(PBS)]. Another 2 ml from the rest of n-octanol layer was taken out and repartitioned in a tube previously containing 3 g PBS and 1.65 ml of n-octanol until consistent partitions of the coefficient values were obtained. All assays were performed in triplicate.

### Partition Coefficients

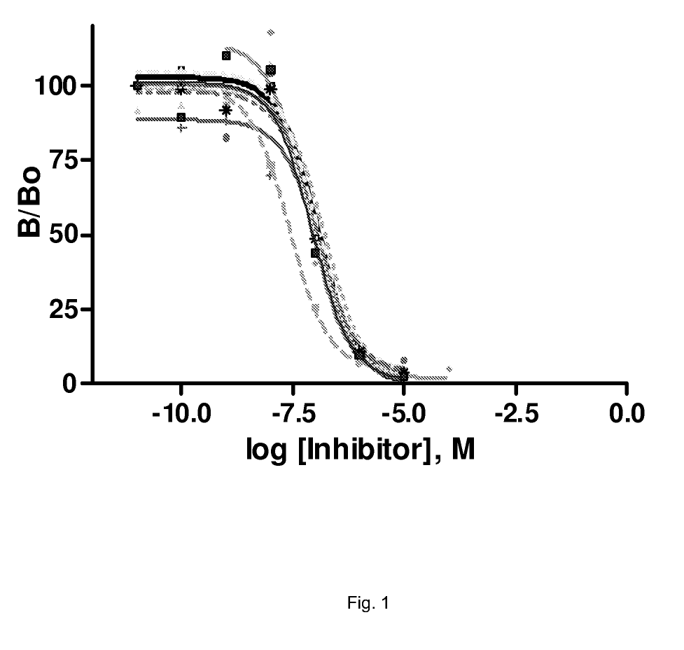

Based on the conventional octanol-water partition measurement, the lipophilicity of [¹²⁵I] 18 was determined in terms of partition coefficients (logP oct). The logP oct of [¹²⁵I] 18 was found at 2.70 and the logP oct values of other dibenzothiazole derivatives were then estimated based on coefficients determined by Hansch and Leo (A substituent Constants for Correlation Analysis in Chemistry and Biology (1979) 1^{st} ed.). As shown in FIG. 1, the logP oct values of these derivatives are between 1 and 3, a range that has been previously proposed for optimal brain uptake (Wu et al. (2005) Curr. Top. Dev. Biol. 70:171-213).

### Quantitation of [125I] 18 in Mice Brain

The radiolabeled ligand [¹²⁵I] 18 eluted from C-18 Sep-Pak™ plus cartridge was dissolved in a mixture consisting of saline (2 ml, 9 mg/ml), ethylene glycol (2 ml), ethanol (0.7 ml), and HCl (0.3 ml, 0.3 nM). Under anesthesia, 0.1 ml of the above solution containing 0.185 MBq of radioactive tracer was administered to the mice through a tail vein injection (Swiss-Webster, n=3, 2 months old). The mice were then sacrificed by a heart puncture at 2, 30, and 60 min postinjection. The brain was rapidly removed, weighed, and counted. The uptake of brain was expressed as percentage of injection dose per gram.

### In Vitro Binding to AD Homogenates

Binding was assayed in 12×75 mm borosilicate glass tubes. For saturation studies, the reaction mixture contained 50 μl of AD homogenates (10-50 μg), 50 μl of [³H]PIB (diluted in PBS, 0.1-1 nM), and 50 μl of cold PIB (10 μM, diluted in PBS containing DMSO (less than 1%)) in a final volume of 500 μl. Nonspecific binding was defined in the presence of 10 μM cold standard PIB in the same assay tubes. For competition binding, the reaction mixture contained 50 μl AD homogenates, inhibitors [10⁻⁵-10⁻¹² mol/L in PBS containing DMSO (less than 1%)], [³H]PIB (in PBS, 0.05 nM in the final mixture), and PBS (10 mM) in a final volume of 500 μl. The resulting mixture was incubated at 37° C. for 1 h, and the bound and free radioligands were separated by rapid vacuum filtration through Whatman GF/B glass filter paper using a Brandel M-24R cell harvest and rapidly washed three times at room temperature with PBS. The filters containing the bound radioactivity were transferred to special vials containing 3 ml of universal scintillation fluid. Vials were counted using Beckman LS6500 multi-purpose scintillation counter. Specific binding was estimated as the difference between total and nonspecific binding. Under the assay conditions, the specifically bound fraction was less than 15% of the total radioactivity. The results were subjected to nonlinear regression analysis using software GraphPad Prism by which K_{d} and Kᵢ values were calculated.

The synthesis of an iodinated compound 4-[2,6′]dibenzothiazolyl-2′-yl-2-iodo-phenylamine (18) and its radiolabeling with ¹²⁵I is described in Scheme 2. Thus, the cold standard compound 18 was first synthesized by treating 10 with ICl in AcOH at room temperature for 18 h. Similarly, compound 10 was also used directly as the precursor for radiolabeling. This synthesis of [¹²⁵I] 18 was achieved through direct radioiodination using sodium [¹²⁵I] iodide in the presence of Chloramine T(ChT). The reaction was monitored by HPLC and went to completion after 3 h. The overall radiochemical yields of [¹²⁵I] 18 were

20-30% after HPLC purification. [¹²⁵I] 18 was obtained with a radiochemical purity over 98% and a specific activity near the theoretical limit (80 TBq/mmol) based on the no-carrier added sodium [¹²⁵I] iodide. The radiochemical identity of [¹²⁵I] 18 was verified by co-elution with the non-radioactive cold standard 18 on HPLC profiles. [¹²⁵I] 18 was stable enough to be kept for up to 8 h at room temperature and for up to 2 months in the refrigerator.

### In Vitro Quantitative Binding Assay Using AD Brain Region Homogenates

The binding affinities of these newly developed compounds for β-amyloid were evaluated using AD brain homogenates and tritiated PIB ([³H]PIB, Amersham), a radioligand previously developed with a high affinity for synthetic Aβ aggregation (Kᵢ=4.3 nM). The postmortem brain tissues were obtained from well-defined AD patients. The gray matter was then carefully separated from white matter at autopsy and kept in −70° C. The fresh frozen gray matter was then homogenized by milling it thoroughly in a mortar in the presence of liquid nitrogen. The homogenates were then prepared in phosphate-buffered saline (PBS, pH 7.4) at a concentration of approximately 400 mg tissue/ml, aliquoted into 1-ml portions, and stored

at −70° C. for future use.

As shown in FIG. 1, the newly developed dibenzothiazole derivatives competed effectively with [³H]PIB binding site(s) on AD homogenates at high affinities, the Kᵢ values of compounds 10-18 are shown in FIG. 1, which showed relatively high binding affinities in the range of 6.8-36 nM. The results indicated that these dibenzothiazole derivatives bind to the same site as PIB does on AD homogenates. According to the in vitro binding assays, functional groups have moderate effects on the binding affinity. Compounds containing electrondonating groups showed slightly higher binding affinity than those containing electron-withdrawing group. For example, the Kᵢ values decreased in the order of 12 (6-F)>11 (6-Cl)>10 (6-H)>PIB (6-OH), consistent with the order of increasing electron-donating capacity. In addition, methylation of the amino group increased the binding affinity. Thus, N,N-dimethylated derivative (compound 17) and N-monomethylated derivatives (compounds 14-16) displayed higher binding affinities than that of the primary amino derivatives (compounds 10-13).

### In Vivo Brain Uptake in Normal Mice

For potential in vivo imaging studies, we radiolabeled compound 10 with ¹²⁵I for brain uptake studies. Following a single iv injection of [¹²⁵I] 18 (0.2 ml, 0.185 MBq), the brain permeability was evaluated in normal mice. The brain radioactivity concentration of [¹²⁵I] 18 was determined at 2, 30, and 60 min postinjection. As shown in Table 1, [¹²⁵I] 18 displayed rapid brain entry at early time intervals. The initial brain uptake was 3.71±0.63% ID/g at 2 min postinjection, a level that is considered for potential clinical imaging studies. The brain radioactivity concentration decreased sharply to 0.78±0.14% ID/g at 30 min and 0.43±0.12% ID/g at 60 min, with a 2-to-30 min ratio of 5. These results indicate that the non-specific binding of [¹²⁵I] 18 was just as low as it rapidly clears from the normal mouse brain in the absence of amyloid deposits.

TABLE 1
Brain uptake in mice (n = 3, % ID/g)

| Compound | 2 min. | 30 min. | 60 min. |
|---|---|---|---|
| [¹²⁵I]18 | 3.71 ± 0.63 | 0.78 ± 0.14 | 0.43 ± 0.12 |

### Example 2

### Development of Novel Amyloid Imaging Agents

We designed and synthesized a series of novel amyloid imaging agents listed in Table 2 (i.e., compounds II-VII).

TABLE 2

| | | |
|---|---|---|
| I PIB 10 mM | Mw: 256 | 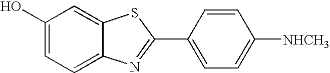 |
| II 10 mM | Mw: 387 | 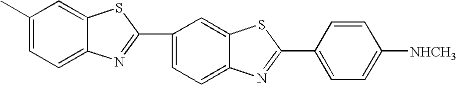 |
| III 10 mM | Mw: 373 | 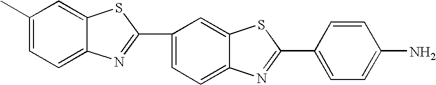 |
| IV 10 mM | Mw: 359 | 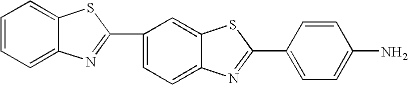 |
| V 10 mM | Mw: 377 | 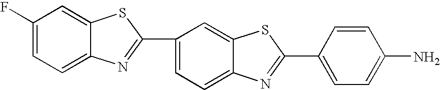 |
| VI 10 mM | Mw: 394 | 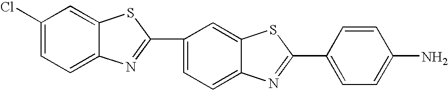 |

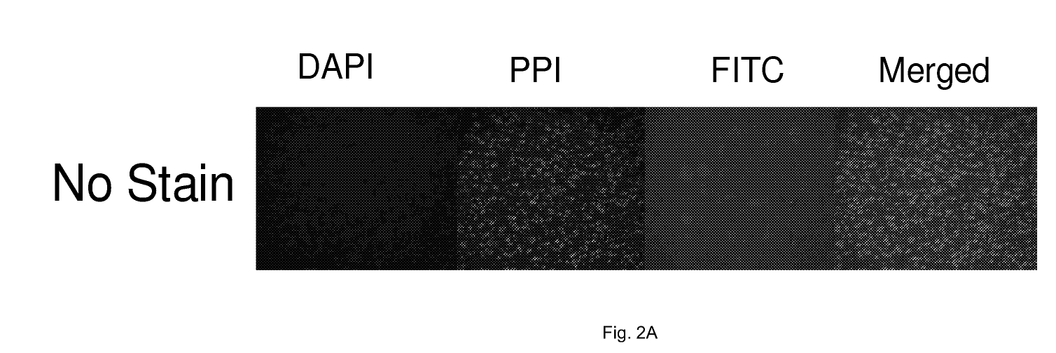

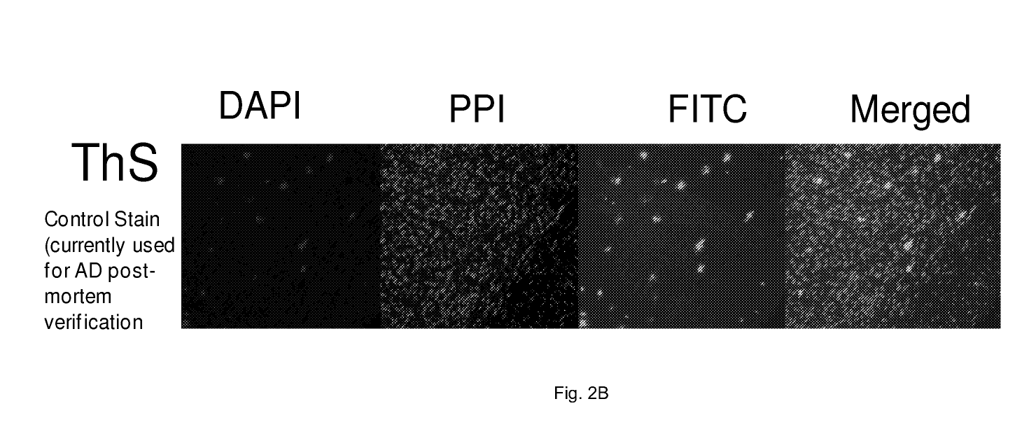

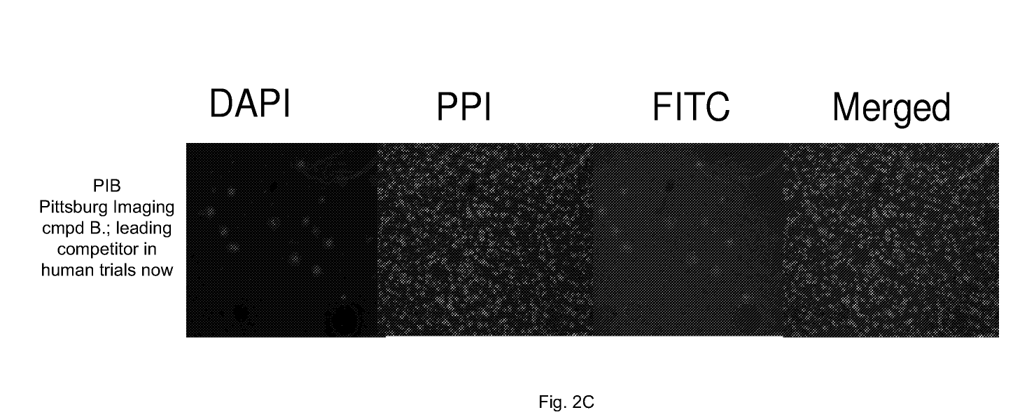

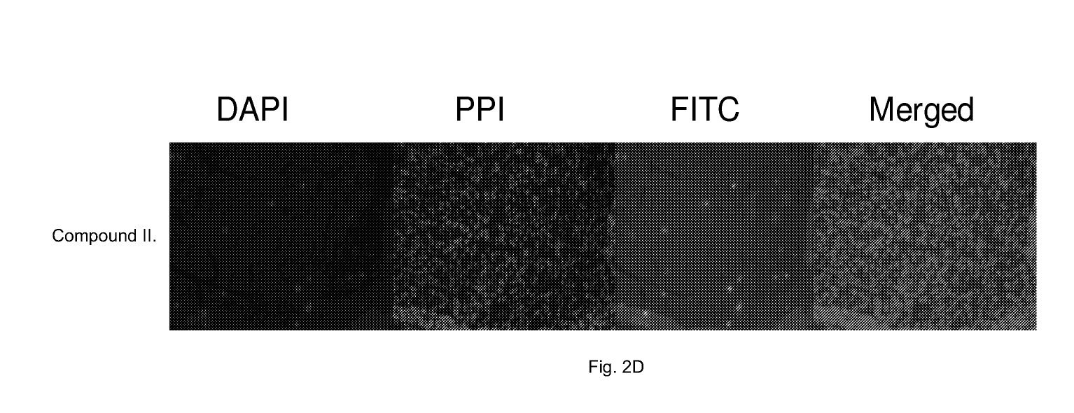

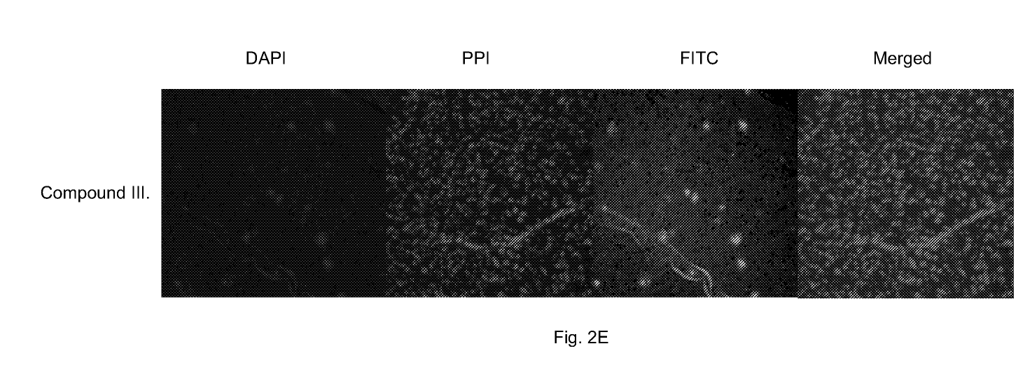

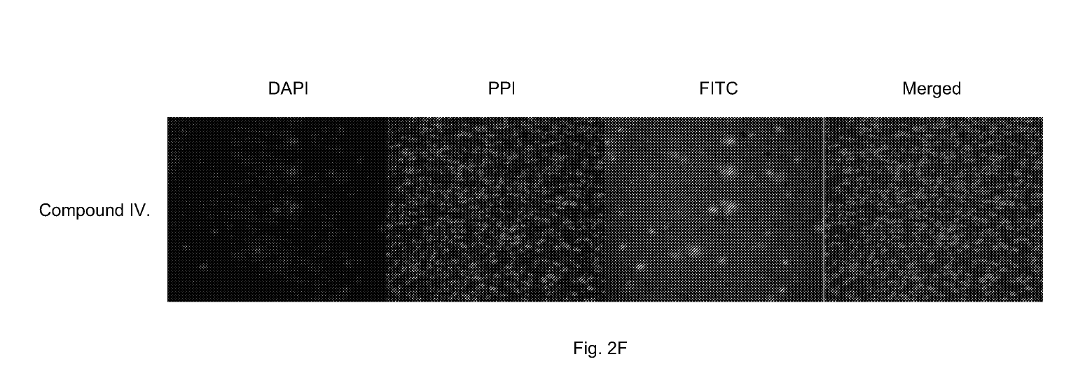

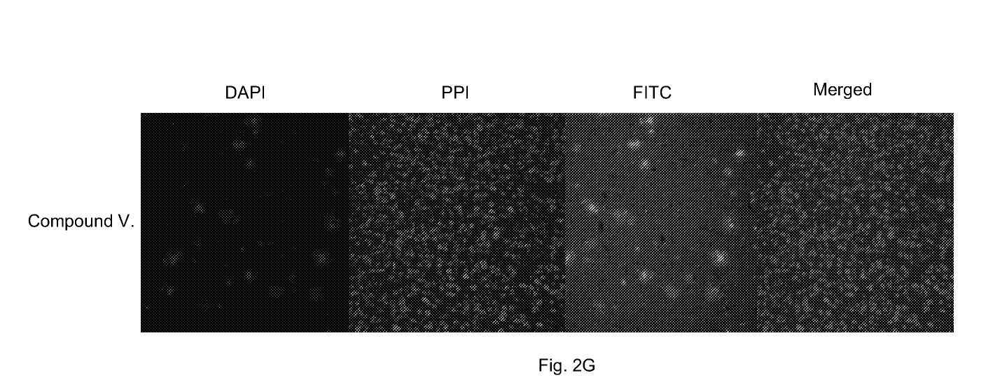

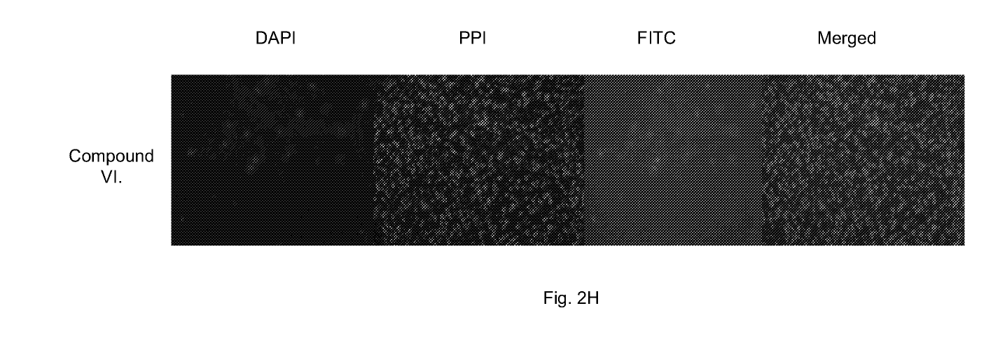

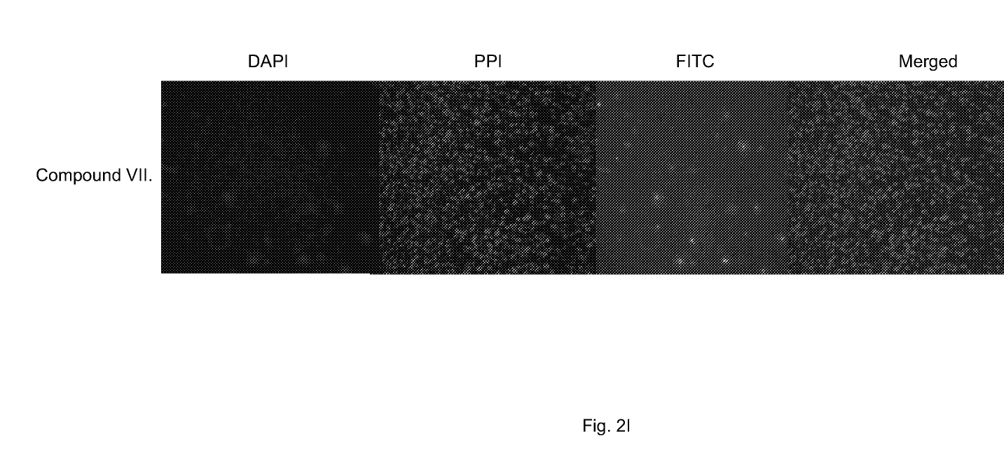

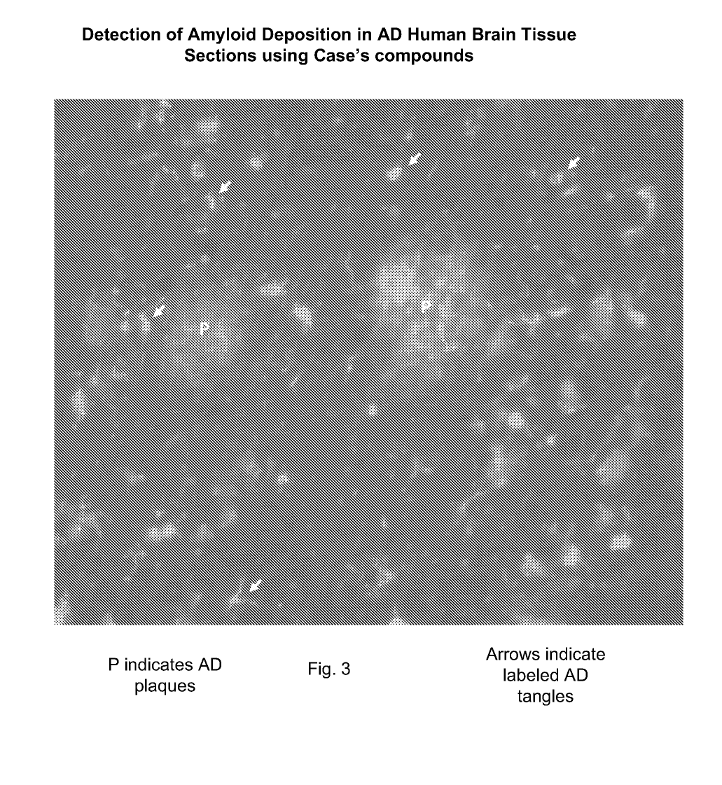

Both in vitro (mouse and human AD brain) and in vivo (mouse, ¹¹C labeled compound, micro-PET) studies of these agents were conducted. The results are shown in FIGS. 2 and 3. The compounds selectively stained amyloid deposits (plaques and tangles) in transgenic mouse models and AD brain tissue sections. A lead agent has been radiolabelled with C-11 for in vivo imaging studies in mice, which readily penetrate the blood-brain barrier.

### In Vitro Staining of Beta-Amyloid

We analyzed six month old APPPS1 mice brain sections and Non-transgenic control brain sections. We counter stained all sections with Propidium Iodide (PPI) (labels cell bodies) and DAPI (nuclei), Thioflavin-S (positive control), labels AD plaques, PIB (“Compound I”) (leading competitor compound in human trials now), “Compounds II-VII”.

### Basic Tissue Staining Procedure

We prepared 0.3 mM and 0.01 mM dilutions of all stains, washed brain sections in PBS solution for 10 min, stained brain sections for 5 min, washed in PBS for 10 min, placed brain sections in propidium iodide for 10 min, washed in PBS for 10 min, and cover-slipped sections and sealed with nail polish.

### Example 3

### In vivo MicroPET studies of [C-11]CIA in Mouse Brain

### Radiosynthesis of [C-11]CIA

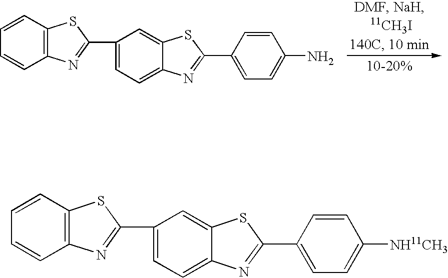

The cyclotron-derived [C-11]carbon dioxide was converted to [C-11]methyl iodide by reduction with lithium aluminum hydride and hydroiodide. The labeled methyl iodide ([C-11]CH₃I) formed was concurrently distilled and trapped in a dry ice-bath cooled 5-mL conical reaction vial containing the 2 mg of precursor, 10 mg sodium hydride in 0.3 mL dimethyl formamide. Trapping was monitored by measuring the activity in the isotope calibrator until the maximal value was attained. The reaction mixture was sealed and heated at 140° C. for 10 minutes in a heating block, cooled to room temperature and diluted with water. The radiolabeled reaction mixture was passed through a C-18 Sep-Pak previously conditioned with ethanol and water. The Sep-Pak was eluted with ethanol and the ethanol solution was loaded on a preparative HPLC (Luna 5μ C18 250×10 mm) column eluting with acetonitrile and water (8:2, v/v) with a flow rate at 4 mL/min. The radioactive fraction containing CIA was collected (retention time 10.5 min), the radiochemical purity of [C-11]CIA was >95% as determined by radio-HPLC. After evaporation of the mobile phase, the residue was re-dissolved in 10% ethanol in saline solution. The solution was filtered through 0.22 μm into a sterile injection flask for injection.

### MicroPET Studies

MicroPET studies were carried out using a Concord R4 microPET scanner (Knoxville, Tenn.) under anesthesia. After a 10 min transmission scan with a Co-57 source, 2 mCi/kg of radiolabelled [¹¹C]CIA was administered to the animal through a tail vein injection, which was immediately followed by dynamic acquisition for up to 100 min.

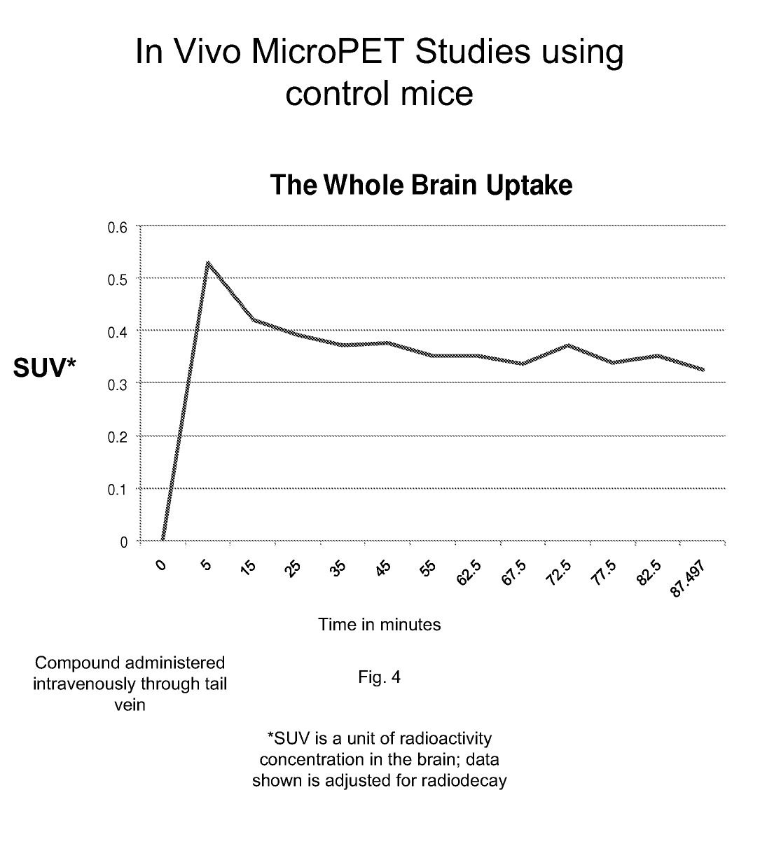

List-mode emission data was analyzed as histograms with 12×5-sec, 12×30-sec, 5×60-sec, and 17×300-sec dynamic frames. A 2-D filtered back projection (FBP) algorithm was used for image reconstruction with a 256×256-pixel resolution per transverse slice. A total of 63 transverse slices was reconstructed with a field of view covering the brain region. Decay correction, attenuation correction and scatter correction were performed during the image histogram and reconstruction processes. The results are plotted in FIG. 4. FIG. 4 shows whole brain uptake at the ¹¹C labeled molecule probe.

Having described the invention, the following is claimed:

1. A molecular probe for use in the detection of amyloid of a subject comprising the general formula: 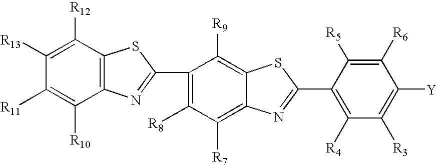
wherein Y is NR₁R₂, OR₂, or SR₂; each R₁-R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof.

2. The molecular probe of claim 1, the benzothiazoles groups of the molecular probe are not quaternary amines.

3. The molecular probe of claim 1, wherein at least one of R₁-R₁₃ includes a radiolabel.

4. The molecular probe of claim 3, wherein the radiolabel of claim 3 is selected from the group consisting of ³H, ¹³¹I, ¹²³I, ¹²⁵I, ¹⁸F, ¹⁹F ¹¹C, ⁷⁵Br, and ⁷⁶Br.

5. The molecular probe of claim 1, wherein R₃-R₁₂ comprise H, and R₁₃ comprises H or an electron donating group.

6. The molecular probe of claim 1, wherein R₃-R₁₂ comprise H, and R₁₃ is selected from the group consisting of H, Cl, F, I, Br, a lower alkyl group, and OCH₃.

7. The molecular probe of claim 5, wherein Y is NR₁R₂.

8. The molecular probe of claim of claim 7, wherein Y is selected from the group consisting of NH₂, NHCH₃, N(CH₃)₂.

9. The molecular probe of claim 1, further comprising a chelating group or a near infrared imaging group.

10. The molecular probe of claim 1, the amyloid comprising amyloid deposits in senile plaques (SPs) and neurofibrillary tangles (NFTs) in a subject's brain tissue.

11. A molecular probe for use in the detection of amyloid in a subject comprising the general formula: 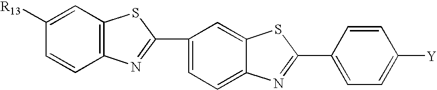
wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof.

12. The molecular probe of claim 11, wherein the molecular probe includes a radiolabel selected from the group consisting of ³H, ¹³¹I, ¹²³I, ¹²⁵I, ¹⁸F, ¹⁹F, ¹¹C, ⁷⁵Br, and ⁷⁶Br.

13. The molecular probe of claim 11, wherein R₁₃ is selected from the group consisting of H, Cl, F, I, Br, a lower alkyl group, and OCH₃.

14. The molecular probe of claim 13, wherein Y is NR₁R₂.

15. The molecular probe of claim of claim 14, wherein Y is selected from the group consisting of NH₂, NHCH₃, N(CH₃)₂.

16. The molecular probe of claim 11, comprising the structure:

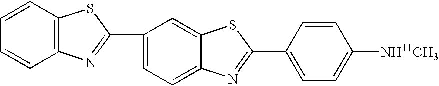

17. The molecular probe of claim 11, further comprising a chelating group or a near infrared imaging group.

18. A method of detecting amyloid in an animal's brain tissue, the method comprising:
(i) administering to the tissue a molecular probe having comprising the general formula: 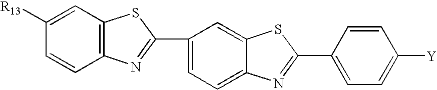
wherein Y is NR₁R₂, OR₂, or SR₂; each R₁, R₂, and R₁₃ independently is selected from the group consisting of H, F, Cl, Br, I, a lower alkyl group, (CH₂)ₙOR′(wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group wherein R′is H or a lower alkyl group), a tri-alkyl tin, a radiolabel, a chelating group, and a near infrared group; or pharmaceutically acceptable salts thereof; and
(ii) detecting the binding of the molecular probe to the animal's brain tissue.

19. The method of claim 18, wherein the molecular probe includes a radiolabel selected from the group consisting of ³H, ¹³¹I, ¹²³I, ¹²⁵I, ¹⁸F, ¹⁹F, ¹¹C, ⁷⁵Br, and ⁷⁶Br.

20. The method of claim 18, wherein R13 is selected from the group consisting of H, Cl, F, I, Br, a lower alkyl group, and OCH₃.

21. The method of claim 18, wherein Y is selected from the group consisting of NH₂, NHCH₃, N(CH₃)₂.

22. The method of claim 18, further comprising a chelating group or a near infrared imaging group.

23. The method of claim 18, the molecular probe being administered in vivo to the animal.

24. The method of claim 23, the molecular probe being detected by an in vivo imaging modality.

25. The method of claim 24, the imaging modality comprising at least one of gamma imaging, Positron Emission Tomography (PET) imaging, micro Positron Emission Tomography (microPET) imaging, Single Photon Emission Computer Tomography (SPECT) imaging, magnetic resonance imaging, magnetic resonance spectroscopy, and near infrared imaging.

26. The method of claim 25, the animal comprising a human or a mouse.

27. The method of claim 26, further comprising the step of administering the molecular probe to the animal intravenously.
